(19) United States
(12) Patent Application Publication
(10) Pub. No.: US 2025/0224410 A1
(43) Pub. Date: Jul. 10, 2025

(54) READY-TO-CONSTITUTE ANALYTICAL PLATFORMS FOR CHEMICAL ANALYSES AND QUANTIFICATION OF ANALYTES IN BIOLOGICAL SAMPLES

(71) Applicant: PinPoint Testing, LLC, Little Rock, AR (US)

(72) Inventors: Jeffrey M. MORAN, Roland, AR (US); Gregory W. ENDRES, Saline, MI (US)

(21) Appl. No.: 18/703,139

(22) PCT Filed: Oct. 21, 2022
(86) PCT No.: PCT/US2022/047455

§ 371 Date: Apr. 19, 2024

Related U.S. Application Data
(60) Provisional application No. 63/270,365, filed on Oct. 21, 2021.

(51) Int. Cl. G01N 33/94; B01L 3/00

(52) U.S. Cl. CPC G01N 33/9486; B01L 3/50851; G01N 33/946; B01L 2200/16; B01L 2300/0829; G01N 2333/924; G01N 2496/00

(57) ABSTRACT

A device for quantifying the concentration of at least one analyte in a liquid test sample, the device comprising: a receptacle or plurality of receptacles, each receptacle configured to hold a liquid sample wherein each receptacle independently comprises: a) a matrix solution, and b) an analyte standard solution comprising at least one analyte standard, wherein the at least one analyte standard corresponds to the at least one analyte being assayed; wherein the matrix solution is separated from the analyte standard solution in said receptacle or plurality of receptacles, and the matrix solution and the analyte standard solution are not in substantial admixture.

### CROSS REFERENCE TO RELATED APPLICATIONS

The present application is an International PCT Application claiming priority to and the benefit of U.S. Provisional Application U.S. 63/270,365, filed on Oct. 21, 2021, the disclosure of which is incorporated herein by reference in its entirety.

### FIELD

The present disclosure relates to devices, kits, and methods for the quantitative analysis of an analyte or analytes in a test sample.

### BACKGROUND

The demand for opioid and other drug testing in both clinical and forensic accredited testing laboratories, ISO17025 and CLIA respectively, has significantly increased with the common, widespread abuse of this class of drugs. Reliable analytical data is important because it helps physicians monitor appropriate use of medication, enables medical examiners to identify specific drugs involved in cause-of-death investigations, allows law enforcement to mitigate drug diversion tactics, and assists rehabilitation centers in deterring abuse. Unfortunately, decreases in discretionary spending and reductions in medical reimbursements require new technologies that increase efficiencies when evaluating standards, quality and process control samples, and unknown human specimens.

It is common practice in human urine analytical toxicology laboratories to incubate samples with beta-glucuronidase enzymes purified from biological sources (red abalone, Halitosis rufescens) or recombinant systems (KURA). This class of enzyme specifically hydrolyzes glucuronic acid metabolites to yield free drugs that can be easily quantified using commercially available certified reference material that is NIST traceable. It is generally thought that enzymatic activity will rapidly degrade glucuronic acid standards used by analytical toxicology laboratories as process controls. As such, this prevents the pre-manufacturing of processing control samples in a ‘ready-to-use’ format, and laboratories build these samples on demand. It is not known if these mixtures can be stabilized at −20° C. or −80° C. or long-term storage.

Analytical toxicology laboratories often detect and quantify multi-drug drug panels that contain unstable analytes. For example, it is well established that heroin and its downstream metabolite 6-acetyl-morphine (6-MAM) rapidly degrade into morphine. Heroin (3,6-Diacetylmorphine) first degrades via deacetylation at the 3′ position into 6-MAM, and through further deacetylation 6-MAM degrades into morphine. This degradation pathway is sensitive to pH and biological matrix effects. For example, basic/acidic extraction protocols commonly performed in analytical toxicology laboratories can degrade heroin or 6-MAM. Biological matrices like human blood can also rapidly degrade heroin, 6-MAM, and other unstable drugs. Analytical toxicology laboratories commonly control for degradation through the use of labeled internal standards, but like that of the process controls that contain glucuronic acid metabolites, the unstable labeled internal standards have to be added to the analytical process at the exact time of use.

It is also common practice for analytical testing laboratories to use authentic reference standards combined with blank, blood, urine, or oral fluid to perform matrix matched, complex testing. This is a challenge because many drugs are not stable in blood, urine or oral fluid for extended periods. For example, cocaine rapidly degrades to benzoylecgonine in blood, and heroin rapidly degrades to 6-MAM and morphine in blood and oral fluid. Therefore, laboratories minimize degradation by building matrix matched standards on demand and incorporate appropriate controls to that account for degradation.

The present disclosure allows for the manufacturing of stable ‘ready-to-use’ test kits that only require the addition of a patient sample, e.g., blood, urine, or saliva, being evaluated to determine what analytes, if any, are in the patient sample.

The present disclosure also allows for the manufacturing of stable spiked matrix samples, e.g., analyte and blood, urine, or saliva, combined for use as a reference standard or quality control sample.

### SUMMARY

The present disclosure provides ready to use devices for analytical assessment and determination of one or more analytes from e.g., urine, blood, and oral fluid.

The present disclosure provides devices for quantifying the concentration of one or a plurality of analytes in a test sample. In various embodiments, the analyte to be measured in a biological sample and standards described herein include: cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl, as well as their internal standards, quality control standards, a process control samples and/or calibration standards.

In various embodiments, the device used to detect and qualitatively and/or quantitatively measure an analyte or plurality of analytes in a liquid test sample, the device comprises a receptacle or plurality of receptacles, wherein each receptacle is configured to hold a liquid sample, wherein each receptacle is independently left empty or independently comprises i) a matrix solution, wherein the matrix solution can include urine, blood, saliva, beta-glucuronidase solution with an enzyme buffer, or any combination thereof, and ii) a standard solution comprising a drug standard selected from a calibration standard, a quality control standard, a process control sample, an internal standard, or any combination thereof; wherein the drug standard further comprises one or more standards comprising an analyte described herein; wherein the matrix solution is separated from the standard solution in said receptacle, and the matrix solution and the standard solution are not in substantial admixture.

The present disclosure additionally provides devices for quantifying the concentration of one or a plurality of analytes in a liquid test sample, wherein the device comprises a multi-well plate where each well independently is left empty or independently comprises i) a matrix solution comprising urine, blood, saliva, beta-glucuronidase solution with an enzyme buffer, or any combination thereof, and ii) a standard solution comprising a drug standard selected from a calibration standard, a quality control standard, a process control sample, an internal standard, or any combination thereof; wherein the drug standard further comprises one or more standards comprising an analyte to be assayed; wherein the matrix solution is separated from the standard solution in said receptacle, and the matrix solution and the standard solution are not in substantial admixture; and wherein at least one of the matrix solution and the standard solution further comprises a tracer to allow for detection of cross-contamination between wells of the device.

The present disclosure also provides kits for quantitative determination of the concentration of one or a plurality of analytes in liquid test samples, comprising a device according to the disclosure and a detailed written description of the specifications of the device.

The present disclosure also provides a system for the detection of one or a plurality of analytes in a liquid sample, for example, a human liquid sample wherein the system comprises
a) a receptacle or plurality of receptacles, each receptacle configured to hold a liquid sample wherein each receptacle independently comprises:
   i) a matrix solution comprising urine, blood, saliva, a beta-glucuronidase comprising an enzyme buffer, or any combination thereof, and
   ii) a standard solution comprising a drug standard selected from the group consisting of a calibration standard, a quality control standard, a process control sample, an internal standard, and a high temperature melting solvent; wherein the drug standard further comprises one or more standards comprising an analyte described herein; wherein the matrix solution is separated from the standard solution in said receptacle or plurality of receptacles, and the matrix solution and the standard solution are not in substantial admixture in said receptacle or plurality of receptacles, and
b) an analyte detection device operable to quantify the amount of the analyte, or plurality of analytes in the liquid sample relative to the amount of a standard solution comprising at least one analyte in a receptacle containing the liquid sample; wherein the amount of the analyte in the liquid sample is detected with an analyte detection device after the standard solution and the matrix solution have mixed with the liquid sample in the receptacle or plurality of receptacles.

The present disclosure also provides for methods of determining the concentration of an analyte or a plurality of analytes in one or a plurality of liquid test samples using a device of the present disclosure comprising the steps of
i) providing a device of the present disclosure, wherein the device comprises a plurality of receptacles configured to hold a liquid sample wherein each receptacle comprises:
   (a) a blank; or
   (b) a matrix solution comprising urine, blood, saliva, a beta-glucuronidase comprising an enzymatic buffer, or any combination thereof, and
   (c) a standard solution comprising a drug standard selected from a plurality of calibration standards and a plurality of internal standards (CS+IS), a plurality of wells containing a plurality of quality control standards (QC), and optionally, wells that are empty (blank), and a plurality of internal standards (QC+IS), a plurality of wells containing a plurality of process control samples, and a plurality of wells containing a plurality of internal standards (IS); wherein the matrix solution is separated from the standard solution and the matrix solution and the standard solution are not in substantial admixture, and wherein the drug standard further comprises one or more standards comprising cocaine, heroin, glucuronide conjugated drugs, methylphenedate, 6-MAM or a 6-MAM metabolite; or
   (d) a matrix solution comprising urine, blood, saliva, a beta-glucuronidase comprising an enzymatic buffer, or any combination thereof, and
   (e) a blank solution;
wherein the matrix solution is separated from the standard solution in said receptacle or plurality of receptacles, and the matrix solution and the standard solution are not in substantial admixture; and
ii. adjusting the temperature of the receptacles to a temperature above freezing, for example, from about 4° C. to about 37° C.;
iii. and mixing the well contents to generate a plurality of well samples;
iv. adding a test sample into a well containing IS;
v. quantitative analysis of the well samples; and
vi. determining the concentration of the analyte or analytes present in the test samples.

### BRIEF DESCRIPTION OF THE FIGURES

FIG. 1 Statistical evaluation showing that the buprenorphine glucuronic acid standard is fully protected and stabilized in ‘ready-to-use’ analytical test kits manufactured with suspended solid state and solid state (direct enzyme interface, and urine matrix interface) technology. Both (A) purified recombinant beta glucuronidase (KURA) and (B) beta glucuronidase purified from abalone tissue show similar results. Complete degradation was estimated by incubating the buprenorphine glucuronic acid standard under optimum reaction conditions for 1 hr. Negative controls were processed like that of mixed kits but excluded the respective beta-glucuronidase enzyme to measure non-specific degradation. All samples were extracted with acetonitrile prior to analysis using a liquid chromatography tandem mass spectrometry method optimized for buprenorphine. Where possible beta-glucuronidase enzyme was removed from suspended solid state, direct enzyme, and urine matrix samples prior to extraction. Acetonitrile was directly added to frozen mixed kits and negative controls to immediately degrade the beta glucuronidase enzyme. Removal of enzyme prior to extraction and the quick addition of acetonitrile limited degradation associated with sample processing and provided a better measurement of storage stability. Peak area of the degradant (free drug) was normalized to the response of codeine which was included to control for day-to-day and analytical variation. Data are presented as average±standard deviation (n=3-4). All experimental groups were manufactured and snap frozen at −20° C. prior to analysis.

FIG. 2 Statistical evaluation showing that the amitriptyline glucuronic acid standard is fully protected and stabilized in ‘ready-to-use’ analytical test kits manufactured with suspended solid state and solid state (direct enzyme interface, and urine matrix interface) technology. Both (A) purified recombinant beta glucuronidase (KURA) and (B) beta glucuronidase purified from abalone tissue show similar results. Complete degradation was estimated by incubating the amitriptyline glucuronic acid standard under optimum reaction conditions for 1 hr. Negative controls were processed like that of mixed kits but excluded the respective beta-glucuronidase enzyme to measure non-specific degradation. All samples were extracted with acetonitrile prior to analysis using a liquid chromatography tandem mass spectrometry method optimized for amitriptyline. Where possible beta-glucuronidase enzyme was removed from suspended solid state, direct enzyme, and urine matrix samples prior to extraction. Acetonitrile was directly added to frozen mixed kits and negative controls to immediately degrade the beta glucuronidase enzyme. Removal of enzyme prior to extraction and the quick addition of acetonitrile limited degradation associated with sample processing and provided a better measurement of storage stability. Peak area of the degradant (free drug) was normalized to the response of codeine which was included to control for day-to-day and analytical variation. Data are presented as average±standard deviation (n=3-4). All experimental groups were manufactured and snap frozen at −20° C. prior to analysis.

FIG. 3 Statistical evaluation showing that the hydromorphone glucuronic acid standard is fully protected and stabilized in ‘ready-to-use’ analytical test kits manufactured with suspended solid state and solid state (direct enzyme interface, and urine matrix interface) technology. Both (A) purified recombinant beta glucuronidase (KURA) and (B) beta glucuronidase purified from abalone tissue show similar results. Complete degradation was estimated by incubating the hydromorphone glucuronic acid standard under optimum reaction conditions for 1 hr. Negative controls were processed like that of mixed kits but excluded the respective beta-glucuronidase enzyme to measure non-specific degradation. All samples were extracted with acetonitrile prior to analysis using a liquid chromatography tandem mass spectrometry method optimized for hydromorphone. Where possible beta-glucuronidase enzyme was removed from suspended solid state, direct enzyme, and urine matrix samples prior to extraction. Acetonitrile was directly added to frozen mixed kits and negative controls to immediately degrade the beta glucuronidase enzyme. Removal of enzyme prior to extraction and the quick addition of acetonitrile limited degradation associated with sample processing and provided a better measurement of storage stability. Peak area of the degradant (free drug) was normalized to the response of codeine which was included to control for day-to-day and analytical variation. Data are presented as average±standard deviation (n=3-4). All experimental groups were manufactured and snap frozen at −20° C. prior to analysis.

FIG. 4 Statistical evaluation showing that the lorazepam glucuronic acid standard is fully protected and stabilized in ‘ready-to-use’ analytical test kits manufactured with suspended solid state and solid state (direct enzyme interface, and urine matrix interface) technology. Both (A) purified recombinant beta glucuronidase (KURA) and (B) beta glucuronidase purified from abalone tissue show similar results. Complete degradation was estimated by incubating the lorazepam glucuronic acid standard under optimum reaction conditions for 1 hr. Negative controls were processed like that of mixed kits but excluded the respective beta-glucuronidase enzyme to measure non-specific degradation. All samples were extracted with acetonitrile prior to analysis using a liquid chromatography tandem mass spectrometry method optimized for lorazepam. Where possible beta-glucuronidase enzyme was removed from suspended solid state, direct enzyme, and urine matrix samples prior to extraction. Acetonitrile was directly added to frozen mixed kits and negative controls to immediately degrade the beta glucuronidase enzyme. Removal of enzyme prior to extraction and the quick addition of acetonitrile limited degradation associated with sample processing and provided a better measurement of storage stability. Peak area of the degradant (free drug) was normalized to the response of codeine which was included to control for day-to-day and analytical variation. Data are presented as average±standard deviation (n=3-4). All experimental groups were manufactured and snap frozen at −20° C. prior to analysis.

FIG. 5. Statistical evaluation showing that suspended solid state technology fully protects and stabilizes 6-MAM in ‘ready-to-use’ analytical test kits manufactured with beta-glucuronidase isolated from abalone. Negative controls were processed like that of mixed kits but excluded the respective beta-glucuronidase enzyme to measure non-specific degradation. Since 6-MAM was directly assayed complete degradation was estimated at the limit of detection (S/N≤10) established for the liquid chromatography tandem mass spectrometry method optimized for 6-MAM detection. All samples were extracted with acetonitrile prior to analysis. Where possible beta-glucuronidase enzyme was removed from suspended solid state, direct enzyme, and urine matrix samples prior to extraction. Acetonitrile was directly added to frozen mixed kits and negative controls to immediately degrade beta glucuronidase enzyme. Removal of enzyme prior to extraction and the quick addition of acetonitrile limited degradation associated with sample processing and provided a better measurement of storage stability. Peak area of 6-MAM was normalized to the response of codeine which was included to control for day-to-day and analytical variation. Data are presented as average±standard deviation (n=3-4). All experimental groups were manufactured and snap frozen at −20° C. prior to analysis.

FIG. 6A is a cross-sectional view of a receptacle and a capsulated standard analyte solution and a separated matrix solution in accordance with various embodiments of the present invention.

FIG. 6B is another cross-sectional view of a receptacle containing a capsulated standard analyte solution and a separated matrix solution and a lid in accordance with various embodiments of the present invention.

FIG. 6C is another cross-sectional view of a receptacle containing a capsulated standard analyte solution and a separated matrix solution and a lid in accordance with various embodiments of the present invention.

FIG. 7A is a cross-sectional view of a receptacle and a capsulated standard analyte solution and a separated matrix solution in accordance with various embodiments of the present invention.

FIG. 7B is another cross-sectional view of a receptacle containing a capsulated standard analyte solution and a separated matrix solution and a lid in accordance with various embodiments of the present invention.

FIG. 7C is another cross-sectional view of a receptacle containing a capsulated standard analyte solution and a separated matrix solution and a lid in accordance with various embodiments of the present invention.

FIG. 8 is a perspective view of a multi-well plate device in accordance with various embodiments of the present invention.

FIG. 9 is an exploded cross-sectional view of the multi-well plate device according to line 9/10 of FIG. 8.

FIG. 9A is a cross-sectional view of a receptacle (a flat bottom well) as shown in FIG. 9 containing a standard analyte solution and a separated matrix solution in accordance with various embodiments of the present invention.

FIG. 10 is another exploded cross-sectional view of the multi-well plate device according to line 9/10 of FIG. 8.

FIG. 10A is a cross-sectional view of a receptacle (a rounded bottom well) as shown in FIG. 10 containing a standard analyte solution and a separated matrix solution in accordance with various embodiments of the present invention.

FIG. 11 shows a line graph demonstrating an ambient temperature time-course study (0 to 75 hr) used to optimize the hydrolysis of several glucuronic acid conjugates commonly used as process controls in the devices of the present disclosure.

FIG. 12 shows a line graph demonstrating a 60° C. time-course study (0 to 2 hr) used to optimize the hydrolysis of several glucuronic acid conjugates commonly used as process controls in the devices of the present disclosure.

FIG. 13 shows a bar graph comparing the equivalency of measurements of known drug concentrations in spiked urine samples between a standard ASB validated procedure when compared to suspended state technology disclosed in the present disclosure.

FIG. 14 shows another bar graph comparing the equivalency of measurements of known drug concentrations in spiked urine samples between a standard ASB validated procedure when compared to suspended state technology disclosed in the present disclosure.

FIG. 15 shows another bar graph comparing the equivalency of measurements of known drug concentrations in spiked urine samples between a standard ASB validated procedure when compared to suspended state technology disclosed in the present disclosure.

FIG. 16 shows another bar graph comparing the equivalency of measurements of known drug concentrations in spiked urine samples between a standard ASB validated procedure when compared to suspended state technology disclosed in the present disclosure.

### DETAILED DESCRIPTION

### Definitions

An “analyte” or “drug analyte” or “drug” (used interchangeably) as used herein refers to a drug, or a drug metabolite, or a medicament, or a synthetic small organic or inorganic molecule of less than 5,000 Daltons. An analyte or “standard analyte solution” (used interchangeably herein) can include a drug, or a drug metabolite, or a medicament, or a synthetic small organic or inorganic molecule of less than 5,000 Daltons or an exemplary base, salt, prodrug, or a solvate of the following molecules: cocaine, heroin, glucuronide conjugated drugs, for example, 11-nor-9-carboxy-Δ9-THC glucuronide, Hydromorphone glucuronide, Morphine-3-beta-D-glucuronide, Oxazepam glucuronide, Buprenorphine-3-beta-D-glucuronide, Codeine-6-beta-D-glucuronide, Lorazepam glucuronide, Norbuprenorphine glucuronide, Oxymorphone-3-beta-D-glucuronide, Tapentadol-3-α-D-Glucuronide, Dihydrocodeine-6-B-D-Glucuronide, 6-B-Naltrexol-3-B-D-Glucuronide, Naloxone-3-B-D-Glucuronide, Amitriptyline-N-B-D-Glucuronide, Temazepam Glucuronide Li salt, methylphenedate, 6-MAM, 6-MAM metabolites, 3-monoacetymorphine, opiates, opioids, benzodiazepines, stimulants, barbiturates, cannabinoids, novel psychoactive compounds, Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, 0-Desmethyltramadol, 0-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl.

An analyte standard is an analyte or “standard analyte solution” (used interchangeably herein) that has a structural modification that permits its detection apart from the analyte itself. For example, an analyte standard is an analyte having at least one atomic substitution in its molecular structure, that permits the detection of both the analyte and its corresponding analyte standard and each can be quantified and assayed.

A “reference standard,” as used herein is a standardized analyte which is used as a measurement base for the analyte to be tested.

Beta-glucuronidase (or β-glucuronidase used interchangeably herein) are members of the glycosidase family of enzymes that catalyze breakdown of complex carbohydrates.

An “internal standard” and “IS” are used interchangeably herein and refer to a reference standard that is modified for detection or is a surrogate reference standard labeled for detection. For example, the internal standard may be a reference analyte standard having at least one atomic substitution in its molecular structure.

A “calibration standard” and “CS” are used interchangeably herein and refer to a reference standard that is used to calibrate an instrument reading with an amount of a measureable analyte.

“Quality control standard” and “QC” as used interchangeably herein, and refer to a reference standard that is 1) obtained or prepared from a source independent of the source of the calibration standard, or 2) is obtained or prepared from a reference standard from the same source as the calibration standard but from a different lot than the reference standard used to prepare the calibration standard, or 3) the quality control standard is used to verify the correctness of a calibration obtained using the calibration standard.

The terms “standard solution” and “standard analyte solution” and “drug analyte solution” are used interchangeably, which include one or more of internal standards, calibration standards and quality control standards.

“Process Control” and “PC” refer to a control sample commonly used by analytical toxicology laboratories to evaluate and monitor a process as part of the analytical procedure. For example, a glucuronide process control sample is commonly evaluated as part of a sample batch to measure the activity of beta-glucuronidase that is added to cleave glucuronic acid metabolites in unknown specimens to the free drug form.

When referring to a well, a receptacle, or vial as “blank” it means that the well or vial does not contain any internal standard, quality control standard, calibration standard, high temperature melting solvent, or the like.

Where a receptacle, i.e. a well from a 96, or 384 microtiter plate or array of tubes, each containing a plurality of wells, a tube or a vial of the device of the one or a present disclosure is described as “containing” a CS, an IS, a QC, or a PC it is to be interpreted as excluding the un-recited standards. For example, where a well is described as containing a CS and an IS, the well does not contain a QC. Similarly, if a well is described as containing an IS, the well does not contain a CS or an QC. If a well is described as containing a QC and an IS, the well does not contain a CS.

“Manufactured to contain” means that components of the test are added to the wells, receptacles, or vials of the device prior to receipt of the device by the end user.

Not permitting the matrix solution to be in substantial admixture with a standard solution as used herein, refers to no contact between the matrix solution and the standard solution such that less than 0.0001% of each of the two solutions are in admixture when stored in a device or receptacle of the present disclosure, preferably 0% of each of the two solutions are in admixture when stored in a device or receptacle of the present disclosure.

“6-MAM” is an abbreviation for 6-monoacetylmorphine.

“6-monoacetylmorphine” metabolites include morphine and 3-monoacetymorphine.

“A,” “an,” “the,” “at least one,” and “one or more” are used interchangeably to indicate that at least one of the items is present; a plurality of such items may be present unless the context clearly indicates otherwise. As used herein a plurality of items can mean two or more of these items. A plurality of wells can mean two or more wells, or even all of the wells of the device.

“Saliva” and “oral fluids” are used interchangeably.

In some descriptions of embodiments of the invention, a well of a multi-well plate is interchangeable with a receptacle or a vial.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps unless clearly specified otherwise in the present disclosure. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

As used herein, a receptacle is defined as a structure that is generally operable to retain a volume of liquid without substantial loss of volume if any at all. The receptacle can be made of any suitable material that is solid or semi-solid and permits the storage or addition of liquid in a volume ranging from 5 microliters to about 500 milliliters, and any volume there between. Suitable receptacles may also be frozen during storage and are generally made of a plastic, ceramic, glass, quartz, or metal material. Exemplary receptacles may include wells of a macro or micro titer multiwell plate, a tube, or a vial.

“A multi-well assay plate” is also referred to as “a multi-well plate”, or a “microtiter plate”, in each, the multi-well assay plate can have a plurality of wells ranging from about 4 to about 1,536 wells, for example, 96 wells or 384 wells, or 768 wells, wherein the wells in each of the multi-well assay plates can be cylindrical with flat or rounded bottoms. The multi-well assay plate can be made of any durable solid material, ranging from plastics commonly used in the manufacture of such plates, for example, polystyrene or polypropylene, but could also include other materials such as ceramics, quartz, glass and other solid materials. The wells of the multi-well assay plate can accommodate any volume commonly employed in the field, for example, ranging from about 5 μL to about 50 mL, and any integer in-between, for example, in a 96 well multi-well assay plate, the internal volume of each well is about 360 μL.

The fully prepared test kit for sale to the customer may be referred to as a product.

The antecedent “about” indicates that the values are approximate. For example, the range of “about 1 mg to about 50 mg” indicates that the values are approximate values. The range of “about 1 mg to about 50 mg” includes approximate and specific values, e.g., the range includes about 1 mg, 1 mg, about 50 mg and 50 mg.

When a range is described, the range includes both the endpoints of the range as well as all numbers in between. For example, “between 1 mg and 10 mg” includes 1 mg, 10 mg and all amounts between 1 mg and 10 mg, for example, 0.1 mg, 2 mg, 5.6 mg, 9.75 mg, and 9.9 mg. Likewise, “from 1 mg to 10 mg” includes 1 mg, 10 mg and all amounts between 1 mg and 10 mg, for example, 0.1 mg, 2 mg, 5.6 mg, 9.75 mg, and 9.9 mg.

The present disclosure provides ready-to-use assay kits and methods for the rapid quantitative analysis of analytes in a test liquid sample while eliminating the need for the end user to prepare standardized solutions of the analytes, calibration standards, quality control standards, internal standards, or process controls.

Adopting a problem solution approach, the present disclosure enables laboratories that conduct drug testing for drugs present in urine or blood samples, that must use B-glucuronidase enzyme to process the samples before testing. This is because many drugs are excreted in urine in a conjugated form (as a glucuronide adduct of the drug). The B-glucuronidase enzyme is needed to remove this adduct to test for the presence of drugs or drug metabolites of interest in the urine or blood sample. The B-glucuronidase enzyme used by the laboratories during testing may be derived from biological sources or via recombinant protein production. Many laboratories use purified B-glucuronidase extracted from the abalone mollusk. A problem with using abalone-based B-glucuronidase is the potential for degradation of analytes of interest if in contact with the drug for an extended period. For example, 6-monoacetyl morphine (6-MAM), used to confirm heroin use, will degrade to morphine in the presence of abalone-derived 6-MAM. Therefore, it is problematic for a drug testing kit, that includes both the 6-MAM standard and B-glucuronidase in contact with one another, because of the significant degradation of 6-MAM to morphine that occurs. This is problem solved by separating the drug to be assayed from the B-glucuronidase solution within the well until the kit is ready to be used, thereby minimizing, or eliminating the degradation. Problems with degradation also occur when B-glucuronidase enzyme, blood, urine, or other biological matrices are in contact with drug analytes. For example, heroin readily degrades to 6-MAM and morphine when combined with blood. The present disclosure provides one or more solutions to these problems, inherent in the present screening and testing methods and assay materials, by providing methods and devices and receptacles that provide a physical barrier between the analyte drug or drugs to be quantified and the matrices which are required to provide a more accurate representation of the actual drug analyte being assayed.

### Devices

One aspect of the present disclosure provides a device that comprises a receptacle or a plurality of receptacles, for example, a tube, or a plurality of tubes, a vial, or a plurality of vials, or a multi-well assay plate comprising a certain number of wells. In some embodiments, the device is manufactured such that each receptacle, i.e. a vial, a tube or a well (within a multi-well plate) independently contains a matrix material (for example, blood, urine or saliva) and a precise, pre-determined quantity of a calibration standard, a precise, pre-determined quantity of a quality control standard, a precise, pre-determined quantity of an internal standard, pre-determined quantity of a process control standard (i.e. analyte standards or drug analyte) or is left blank.

Another aspect the present disclosure provides a device that comprises a receptacle or a plurality of receptacles, for example, a tube, or a plurality of tubes, a vial, or a plurality of vials, or a multi-well assay plate comprising a certain number of wells. In some embodiments, a device is manufactured such that each receptacle, i.e. a vial, a tube or a well (within a multi-well plate) independently contains a matrix solution with one or more deconjugation enzymes, a precise, pre-determined quantity of a calibration standard, a precise, pre-determined quantity of a quality control standard, a precise, pre-determined quantity of an internal standard, pre-determined quantity of a process control standard (i.e. analyte standards or drug analyte), or is left blank, wherein the matrix solution and the standards solution (i.e. analyte standards or drug analyte) are not in contact and are not in substantial admixture with one another. In yet another aspect the present disclosure provides a device that comprises a receptacle or a plurality of receptacles, for example, a tube, or a plurality of tubes, a vial, or a plurality of vials, or a multi-well assay plate comprising a certain number of wells. In some embodiments, a device is manufactured such that each receptacle, i.e. a vial, a tube or a well (within a multi-well plate) independently contains one or more of a matrix solution comprising at least one of deconjugation enzymes, urine, blood, or saliva, beta-glucuronidase and an enzyme buffer solution, and the device further comprises a standard solution containing standards of the analyte (i.e. analyte standards or drug analyte) or a plurality of analytes in a separate solution that contains a precise, pre-determined quantity of a calibration standard, a precise, pre-determined quantity of a quality control standard, a precise, pre-determined quantity of an internal standard, pre-determined quantity of a process control standard (i.e. analyte standards or drug analyte), or is left blank, wherein the matrix solution and the standards solution are not in contact and are not in substantial admixture with one another. In some embodiments, the matrix solution and the standards solution are each frozen and are separated from one another by a gap of air within the receptacle or have a physical barrier between the two solutions for example, either the matrix solution or the analyte standards (or drug analyte) are contained within a biodegradable or aqueous solution degradable capsule that prevents their admixture. Another aspect of the present disclosure provides a device that comprises chemical tracers that enable the detection of cross-contamination that may occur during use.

In another aspect the present disclosure provides a device that comprises a multi-well assay plate comprising a certain number of wells wherein the device is manufactured such the wells independently contain a matrix solution and a standard solution that includes one or more of precise, pre-determined quantity of a calibration standard, a precise, pre-determined quantity of a quality control standard, a precise, pre-determined quantity of an internal standard, pre-determined quantity of a process control standard, (i.e. analyte standards or drug analyte) or is left blank, wherein the matrix solution and the standard analyte containing solution are not contacting one another and are separated from each other by air or a physical barrier, for example, a solid or liquid that prevents the matrix solution and standard solution from coming into contact with each other. Another aspect of the present disclosure provides a device that comprises chemical tracers that enable the detection of cross-contamination that may occur during use.

In one embodiment, the multi-well assay plate is a 12-well plate. In one embodiment, the multi-well assay plate is a 48-well plate. In another embodiment the multi-well assay plate is a 96-well plate. In yet another embodiment, the multi-well assay plate is a 384-well plate. In still another embodiment the multi-well plate is a 1536-well plate. In various embodiments, the multi-well assay plate can be manufactured using any chemically compatible plastics and solid substrates. In some embodiments, the multi-well assay plate is suitable for in-situ fluorescence or chemiluminescence analysis. In various embodiments, the multi-well assay plate is biologically inert, non-toxic, compatible with various aqueous and organic solvents, does not leach any chemical residues, and does not interfere with the quantitative analysis of the well samples.

For certain analyses the volume of the test sample is greater than the volume of a well of a multi-well plate. Therefore, another aspect of the invention provides for a device that comprises containers such as vials or tubes which are used in place of the wells of a multi-well plate. In some embodiments of the invention the containers are supported by a vial tray. In one embodiment of the vials tray it has from about 1 to 300 vials. In one embodiment the vial tray is a 28-position vial tray. In one embodiment, the vial tray is a 54-position vial tray. In one embodiment, the vial tray is a 108-position vial tray. In one embodiment, the vial tray is a 216-position vial tray.

It should be understood that the contents of each of the wells or vials in a device are independent of each other such that a percentage of the wells or vials may contain only an internal standard, a percentage of the wells or vials may contain a calibration standard and an internal standard, a percentage of wells may be empty, etc., depending upon the specific assay to be performed. Further, it should be understood that although an embodiment is described in terms of a multi-well plate device, unless clearly stated otherwise, the same components and configurations as described for multi-well plate can be used where the device is a vial tray or other type of container, scaling up as appropriate.

In one embodiment of the invention, the device is a multi-well plate comprising wells that contain within each well a matrix solution separated from a standard solution that contains one or more calibration standards and one or more internal standards (CS+IS), wells that contain the internal standard only (IS), wells that contain a quality control standard and an internal standard (QC+IS), wells contain a process control standard and an internal standard (PC+IS), and wells that are empty (blank). In yet another embodiment the device is a vial tray comprising vials that contain a calibration standard and an internal standard (CS+IS), vials that contain an internal standard only (IS), vials that contain a quality control standard and an internal standard (QC+IS), vials that contain a process control standard and an internal standard (PC+IS), and vials that are empty (blank).

In some embodiments the internal standard is a reference standard wherein the reference standard has at least one atomic substitution in its molecular structure.

In some embodiments, the atomic substitution is an isotope of the substituted atom. In some embodiments, the substituted atom is hydrogen and the isotope is deuterium. In some embodiments, the substituted atom is carbon-12 and the isotope is carbon-13. In some embodiments, the substituted atom is nitrogen-14 and the isotope is nitrogen-15. In some embodiments, the substituted atom is oxygen-16 and the isotope is oxygen-18.

In some embodiments, the atomic substitution is a hydrogen atom substituted with atom that is not carbon or nitrogen. In some embodiments, the other atom is fluorine.

The devices of the present disclosure may be configured to quantify a panel of analytes.

Another embodiment of the present disclosure is a device that incorporates tracers that allow for detection of cross-contamination between the wells of a multi-well plate that may occur during performance of an assay. A tracer can be, for example, a uniquely labeled standard or a standard or other reagent that is not the same as another component in the assays and does not interfere any of the components in the assay or interfere with the detection of the analytes. The tracer must also effectively track along with the analytes of interest, for example, a tracer must have the same or similar extraction efficiency of the analytes of interest. Particularly suitable tracers are the same compound as an internal standard but differentially labeled. For example, if a morphine-d6 derivative is used as an internal standard, a differentially labeled morphine derivative such as ¹³C-labeled morphine-derivative, a morphine-d7 derivative, a ¹³C-labeled morphine-d6 derivative and a ¹³C labeled morphine-d7 derivative would be suitable tracers. In one embodiment of the device of the present disclosure is a 96-well plate (e.g. a 12 column×8 row 96-well plate) comprising four different tracers which are configured in the wells as follows: the first tracer is added to every other row consisting of 12 individual wells. The second tracer is added to the rows that were previously skipped. Similarly, the third tracer is added to every other column consisting of 8 individual wells. The fourth tracer is added to the columns that were previously skipped. One of skill in the art based on the present disclosure could construct other tracer configurations for use in wells of a multi-well plate. For example, some configurations may allow only for detection of vertical contamination. Other configurations may allow only for detection of horizontal contamination. And yet other configurations would allow for detection of both vertical and horizontal contamination depending on the placement of the tracers relative to the test samples. The devices of the present disclosure that are manufactured to contain tracers to allow the end user, once the assay is complete, to evaluate each well to determine which tracers are present. Detection of a tracer in a particular well that was not originally in the well indicates cross contamination.

The present disclosure provides devices or systems for detection of one or more analytes in a liquid sample, (for example, a human liquid sample, for example, a blood sample (for example, a whole blood, plasma or serum sample), a urine sample, a saliva sample, an ascites sample, a cerebrospinal fluid sample). The devices or systems for detection of the analyte or plurality of analytes also includes a matrix solution and a standard solution, wherein the matrix solution and standard solution are not in contact with each other until the device or system is ready for assaying a liquid sample. At the time of assaying the liquid test sample, the matrix solution and standard solution are permitted to come into contact by heating the device, or mixing the contents of the wells or receptacles of the device, to bring the matrix solution and standard solution to at least partial admixture. Before, during, or after the time when the matrix solution and standard solution are brought into contact with each other, the test liquid sample is introduced into the wells or receptacles of the device and is in contact with both the matrix solution and the standard solution.

The matrix solution comprises one or more of blood, urine, saliva, a B-glucuronidase enzyme combined with an enzyme buffer solution, or a combination thereof. In some embodiments, the B-glucuronidase enzyme may be in a buffer solution that may comprise a weak base and a weak acid. In some embodiments, the weak base is an acetate buffer. In some embodiments the weak acid is formic acid, acetic acid, benzoic acid, oxalic acid, hydrofluoric acid, nitrous acid, sulfurous acid, phosphoric acid, or combinations thereof. In some embodiments the week acid is acetic acid.

In some embodiments the matrix solution comprises a B-glucuronidase comprising an enzymatic buffer (B-glucuronidase solution) and comprises 500 units to 100,000 units of B-glucuronidase. In other embodiments, the B-glucuronidase solution comprises 1,000 units to 50,000 units of B-glucuronidase. In yet other embodiments the B-glucuronidase solution comprises 1,000 units to 40,000 units of B-glucuronidase. In still other embodiments the B-glucuronidase solution comprises 15,000 units to 30,000 units of B-glucuronidase. In another embodiment the B-glucuronidase solution comprises 10,000 units to 20,000 units of B-glucuronidase. In one embodiment the B-glucuronidase solution comprises 4,000 units. In still other embodiment the B-glucuronidase solution comprises 5,000 units. In yet other embodiment the B-glucuronidase solution comprises 6,000 units. In still other embodiment the B-glucuronidase solution comprises 7,000 units. In yet another embodiment the B-glucuronidase solution comprises 8,000 units. In still another embodiment the B-glucuronidase solution comprises 9,000 units. In another embodiment the B-glucuronidase solution comprises 10,000 units. In some embodiments comprising the beta-glucuronidase solution, the pH of the enzymatic reaction buffer ranges from 3.5 to 7.5. In other embodiment the pH the enzymatic buffer ranges from 4.0 to 7.5. In yet another embodiment the pH the enzymatic buffer ranges from 5.0 to 7.5.

In some embodiments of the invention the beta-glucuronidase solution is essentially free of sulfatase. In some embodiments of the present disclosure the beta-glucuronidase solution comprises beta-glucuronidase isolated from: Patella vulgata, Helix aspersa, Helix pomatia, Abalone, and mammalian liver. In yet another embodiment the beta-glucuronidase is isolated from abalone or from a purified recombinant source.

In some embodiments the matrix solution comprises 0.01 mL to 100 mLs of urine.

In some embodiments the matrix solution comprises 0.01 mL to 100 mLs of blood.

In some embodiments the matrix solution comprises 0.01 mL to 100 mLs of saliva.

In some embodiments the matrix solution comprises 0.01 mL to 100 mLs of a solution containing a beta-glucuronidase enzyme and an enzyme buffer solution.

In some embodiments the matrix solution comprises 0.01 ml, to 100 mLs of a solution containing a beta-glucuronidase enzyme and an enzyme buffer solution and one of blood, urine, and saliva.

In some embodiments, in the manufacture of the device, (e.g. a multi-well plate with a plurality of wells, or a vial, or a vial tray comprising a plurality of vials, or a tube, or an array of tubes, the matrix solution comprises, beta-glucuronidase in the enzymatic buffer, and one of urine, blood or the saliva. The matrix solution is then added to one or more receptacles, and the device is frozen in the range of about −80° C. to about −4° C. In other embodiments the device comprising a plurality of receptacles is frozen at about −80° C., about −75° C., about −70° C., about −65° C., about −60° C., about −55° C., about −50° C., about −55° C., about −50° C., about −45° C., about −40° C., about −35° C., about −30° C., about −25° C., and about −20° C. In some embodiment the device comprising a plurality of receptacles is frozen in the range of −80° C.-20° C. In still other embodiments the device comprising the plurality of receptacles is frozen at −80° C., −75° C., −70° C., −65° C., −60° C., −55° C., −50° C., −55° C., −50° C., −45° C., −40° C., −35° C., −30° C., −25° C., or −20° C.

Together with a frozen matrix solution, in some embodiments, the device and systems of the present disclosure also includes a sample of a solution comprising a standard analyte solution that is separated from the matrix solution. In some embodiments, the standard analyte solution containing at least one analyte standard, for example, one or more of a calibration standards and/or one or more internal standards, and/or one or more of process control standards, and/or one or more quality control standards is also added to the device and plurality of receptacles and frozen above or below the later of matrix solution, wherein the matrix solution and the standard analyte solution (the terms “standard solution” and “standard analyte solution” and “drug analyte solution” are used interchangeably) are not in contact with each other, for example, the two solutions are separated by a physical barrier, for example a solid, semi-solid or liquid barrier or each layer is frozen with a barrier of air between the two frozen matrix and standard solution layers.

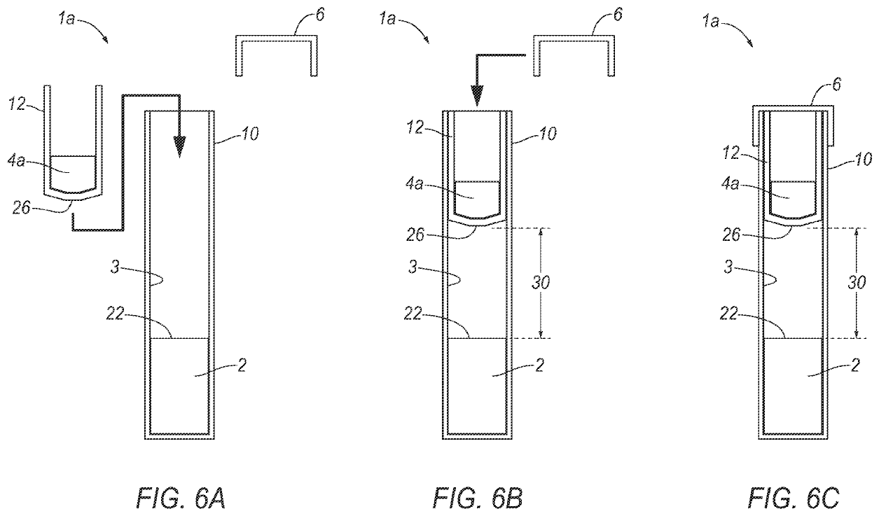

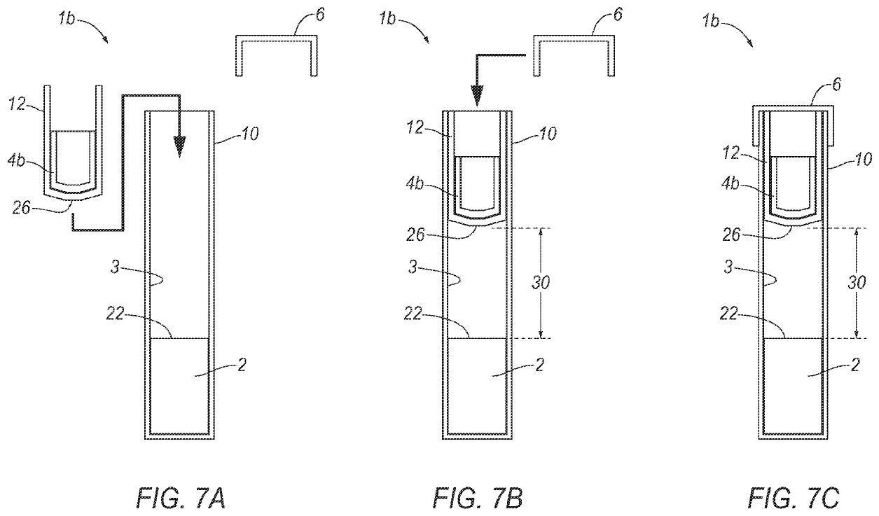

In some embodiments the physical barrier between the frozen matrix solution and the drug analyte solution containing the analyte standards is a gelatin or plant polysaccharide material, such as carrageenan, starch or cellulose material. In some embodiments the physical barrier between the frozen matrix solution and the analyte standards solution is a gelatin or plant polysaccharide such as carrageenan, starch or cellulose in the form of one half of an empty oral capsule shell. In some embodiments the standard(s) may be contained within the empty capsule shell, either in the form of a frozen analyte standard solution or as the standards adsorbed as a substantially dried layer disposed on the internal (lumen) surface of a capsule or part thereof. See FIGS. 6A-7C.

The present disclosure provides devices or systems for detection of a drug analyte or drug medicament or a narcotic, or opioid drug etc or metabolites thereof, which comprise a standard solution comprising of one or multiple drug analytes.

In some embodiments the standard analyte solution comprises a calibration standard. In other embodiments the standard analyte solution comprises a quality control standard. In other embodiments the standard analyte solution comprises a process control standard. In yet other embodiments the standard analyte solution comprises an internal standard. In yet other embodiments, the standard analyte solution contains one or more of an internal standard, a calibration standard, a quality control standard, and a quality control standard.

In an embodiment of a device of the invention, the device is a vial tray comprising a plurality of vials or a multi-well plate comprising wells that contain a standard analyte solution comprising an drug standard, wherein the standard analyte solution comprises one or more of a calibration standard and internal standard (CS+IS), wells that contain an internal standard only (IS), wells that contain a quality control standard and an internal standard (QC+IS), wells that contain a process control standard and an internal standard (PC+IS) wells, and wells that are empty (blank) wherein the standard analyte solution further comprises one or more standards comprising one or more analyte standards.

In an embodiment of a device of the invention, the device is a multi-well plate comprising wells that contain a standard solution comprising a analyte standard selected from the group consisting of: a calibration standard and internal standard (CS+IS), wells that contain an internal standard only (IS), wells that contain a quality control standard and an internal standard (QC+IS), wells that contain a process control standard and an internal standard (PC+IS) wells, and wells that are empty (blank) wherein the drug standard further comprises one or more standards comprising cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl. In some embodiments, the standard analyte solution comprises a 6-MAM metabolite standard.

In some illustrative embodiments comprising a multi-well plate (for purposes of convenience, the exemplary device may be illustrated using a multi-well plate containing a plurality of wells, but other devices are equivalent, wherein a plurality of vials or tubes, each replicating the function of a multi-well plate well, as such all of these wells, vials and tubes are defined as receptacles for the purpose of illustration) may include, except for the blank well, some wells contain a matrix solution, for example, a matrix solution, comprising blood, urine, or saliva, alone or in combination with a solution comprising beta-glucuronidase and an enzyme buffer solution, or combinations thereof, wherein the matrix solution is separated from the standard analyte solution and the matrix solution and standard analyte solution are not in substantial admixture. In some embodiments in which the device, or systems contain a matrix solution comprising a beta-glucuronidase and an enzyme buffer solution, the amount of 3-glucuronidase per well may vary depending for example, on the type, amount, and/or concentration of sample that will be added to the plate by the end user. The amount of 3-glucuronidase can be varied in different wells or ascertained empirically for specific drug-conjugate species. Although f-glucoronidase is exemplified as an optional component of the matrix solution, other drug modifying enzymes, such as deconjugation enzymes found naturally in the liver and kidneys of humans are also contemplated as optional additions to the matrix solution.

Therefore, in some embodiments of the present disclosure, the device may comprise a standard analyte solution comprising an analyte standard selected from the group consisting of a calibration standard, a quality control standard, a process control, calibration standards, and internal standard, wherein the analyte standard further comprises one or more standards comprising an analyte disclosed herein. In one illustrative embodiment, the analyte standard is an isotopic variant of the analyte (for example, a drug, or medicament etc) to be assayed, for example, the analyte standard can include one or more of an isotopic variant of conjugated drugs, methylphenedate, 6-MAM, a 6-MAM metabolite, or any mixture thereof.

In an embodiment of the device, and systems described herein, one or more calibration standards comprising an analyte standard, wherein the analyte standard is an isotopic variant of the analyte to be assayed, for example, the calibration standard can include one or more of an isotopic variant of a drug selected from: cocaine, heroin, glucuronide conjugated drugs, methylphenedate, 6-MAM, a 6-MAM metabolite, or any mixture thereof.

In an embodiment of the device, and systems described herein, one or more quality control standard comprising an analyte standard, wherein the analyte standard is an isotopic variant of the analyte to be assayed, for example, the one or more quality control standard can include one or more of an isotopic variant of cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl, 6-MAM, a 6-MAM metabolite, or any mixture thereof.

In an embodiment of the disclosed system for detection, the receptacles or plurality of receptacles comprise of internal standards, quality control samples, which comprise an analyte standard that is an isotopic variant of one or more analytes described herein. For example, the receptacles or plurality of receptacles comprise of internal standards, quality control samples, which comprise an isotopic variant of one or more analytes selected from cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl, glucuronide conjugated drugs, methylphenedate, 6-MAM, a 6-MAM metabolite or any combination thereof.

In one embodiment of the invention, the system for detection comprises a receptacle or a plurality of receptacles that contain, except for the blank sample, a matrix solution comprising urine, blood, saliva, a beta-glucuronidase comprising an enzyme buffer, or any combination thereof in the receptacles, and a standard solution comprising an analyte standard comprising a plurality of calibration standard and a plurality of internal standard (CS+IS), wells that contain an internal standard only (IS), wells that contain a quality control standard and an internal standard (QC+IS), wells that contain a process control standard and an internal standard (PC+IS), wells that contain one or a plurality of tracers, and optionally, wells that are empty (blank), wherein the matrix solution is separated from the one or more standards and the matrix solution and the one or more standards are not in substantial admixture.

In one embodiment of the invention, the system for detection comprises a receptacle or a plurality of receptacles that contain, except for the blank sample, a matrix solution comprising urine, blood, saliva, a beta-glucuronidase comprising an enzyme buffer, or any combination thereof in the receptacles, and a standard solution comprising an analyte standard comprising a plurality of calibration standards and a plurality of internal standards (CS+IS), wells that contain an internal standard only (IS), wells that contain a quality control standard and an internal standard (QC+IS), wells that contain a process control standard and an internal standard (PC+IS), wherein the analyte standard further comprises one or more standards comprising an isotopic variant of the analyte to be assayed. In some embodiments, the analyte standard comprises one or more isotopic variants of cocaine, heroin, glucuronide conjugated drugs, methylphenedate, 6-MAM or a 6-MAM metabolite. In this embodiment, the matrix solution comprises beta-glucuronidase comprising an enzyme buffer, and the matrix solution and the standard solution are separated by a volume of air or is physically separated with a solid, semi-solid or liquid barrier that does not permit substantial admixture of the two layers.

In one embodiment of the invention, the system for detection comprises a receptacle or a plurality of receptacles that contain, except for the blank sample, a matrix solution comprising urine, blood, saliva, a beta-glucuronidase comprising an enzymatic buffer, or any combination thereof in the receptacles, and a standard solution comprising an analyte standard selected from the group consisting: wells that contain a calibration standard and an internal standard (CS+IS), wells that contain an internal standard only (IS), wells that contain a quality control standard and an internal standard (QC+IS), wells that contain a process control standard and an internal standard (PC+IS), wells that contain one or a plurality of tracers, and optionally, wells that are empty (blank), wherein the matrix solution is separated from the standard solution and matrix solution and standard solution are not in substantial admixture.

In one embodiment of the invention, the system for detection comprises a receptacle or a plurality of receptacles that contain, except for the blank sample, a matrix solution comprising urine, blood, saliva, a beta-glucuronidase comprising an enzyme buffer, or any combination thereof in the receptacles, and a standard solution comprising an analyte standard selected from the group consisting: wells that contain a calibration standard and an internal standard (CS+IS), wells that contain an internal standard only (IS), wells that contain a quality control standard and an internal standard (QC+IS), wells that contain a process control standard and an internal standard (PC+IS), wells that contain one or a plurality of tracers, and optionally, wells that are empty (blank), wherein the matrix solution is separated from the one or more standards and the matrix solution and the one or more standard are not in substantial admixture.

In one embodiment of the invention, the system for detection comprises a receptacle or a plurality of receptacles that contain, except for the blank sample, a matrix solution comprising urine, blood, saliva, a beta-glucuronidase comprising an enzymatic buffer, or any combination thereof in the receptacles, and a standard solution comprising an analyte standard selected comprises wells that contain a calibration standard and an internal standard (CS+IS), wells that contain an internal standard only (IS), wells that contain a quality control standard and an internal standard (QC+IS), wells that contain a process control standard and an internal standard (PC+IS), wells comprising a matrix solution, and optionally, wells that are empty (blank), wherein the matrix solution is separated from the standard solution and the matrix solution and the standard solution are not in substantial admixture.

In one embodiment of the invention, the system for detection comprises a receptacle or a plurality of receptacles that contain, except for the blank sample, a matrix solution comprising urine, blood, saliva, a beta-glucuronidase comprising an enzyme buffer, or any combination thereof in the receptacles, and a standard solution comprising a drug standard selected from the group consisting: wells that contain a calibration standard and an internal standard (CS+IS), wells that contain an internal standard only (IS), wells that contain a quality control standard and an internal standard (QC+IS), wells that contain a process control standard and an internal standard (PC+IS), wells comprising a matrix solution, and optionally, wells that are empty (blank), wherein the matrix solution is separated from the standard solution and the matrix solution and the standard solution are not in substantial admixture. In exemplary embodiments, the system for detection comprises a receptacle or a plurality of receptacles that contain, except for the blank sample, a matrix solution comprising beta-glucuronidase enzyme and an enzyme buffer solution, separated from a standard solution containing analyte standards for one or more analytes selected from cocaine, heroin, glucuronide conjugated drugs, methylphenedate, 6-MAM or a 6-MAM metabolite, wherein the matrix solution and the standard solution are both frozen and are separated from a layer of air, such that the matrix solution and the standard solution are not in contact and there is no admixture between the two solutions.

In the present disclosure, the separation of the matrix solution and the standard solution are each independently frozen layers in the same receptacle.

In the present disclosure, the separation of the matrix solution and the standard solution is by a barrier comprising a solid material or a semi solid material. In some embodiments, the solid or semi solid material is a high melting solvent, paper, plastic, wax, or nanoparticles.

In the present disclosure, the separation of the matrix solution and the standard solution is by suspending the solid material or a semi solid material between the matrix solution and the standard solution. In some embodiments, the solid or semi solid material is paper, plastic, wax, or nanoparticles.

In some embodiments the solid or semi solid material melts at a temperature ranging from about 10° C. to about 80° C., or from about 10° C. to about 70° C., or about 10° C. to about 60° C. In other embodiments, the solid or semi solid material melts at a temperature ranging from 10° C. to 80° C., or from 10° C. to 70° C., or 10° C. to 60° C.

In other embodiments the solid or semi solid material melts at about 10° C., 15° C., 20° C., 25° C., about 30° C., about 35° C., about 40° C., about 45° C., about 50° C., about 55° C., about 60° C., about 65° C., about 70° C., about 75° C., or about 80° C. In still another embodiment, the solid or semi solid material melts at 10° C., 15° C., 20° C., 25° C., 30° C., 35° C., 40° C., 45° C., 50° C., 55° C., 60° C., 65° C., 70° C., 75° C., or 80° C.

One embodiment of the invention is a device for quantifying the concentration of an analyte as described herein. In some exemplary embodiments, the analyte is one or more of: cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl, glucuronide conjugated drugs, methylphenedate, 6-MAM or a 6-MAM metabolite. The device comprises a matrix solution comprising at least one of urine, blood, saliva and beta-glucuronidase and an enzyme buffer solution. The device is manufactured such that the matrix solution and the standard analyte solution are each frozen and separated by a volume or layer of air, such that the matrix solution and the standard solution containing one or more analyte standards comprising one or more isotopic variants of an analyte to be measured and assayed in a liquid test sample.

In some embodiments, an illustrative device comprises: a receptacle or plurality of receptacles, each receptacle configured to hold a liquid sample wherein each receptacle independently comprises: a) a matrix solution comprising urine, blood, saliva, a beta-glucuronidase enzyme comprising an enzyme reaction buffer, or any combination thereof, and b) a standard analyte solution comprising an analyte standard selected from the group consisting of: a calibration standard, a quality control standard, a process control, an internal standard. In various related embodiments, the analyte to be measured in a liquid test sample, for example, a human liquid test sample, selected from a blood test sample, a urine test sample, a saliva test sample. The matrix solution is a beta-glucuronidase enzyme comprising an enzyme reaction buffer, and the assayed analyte to be measured in the liquid test sample, and the analyte standard (isotopic variant) comprises: cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl, glucuronide conjugated drugs, methylphenedate, 6-MAM or a 6-MAM metabolite; wherein the matrix solution is separated from the standard solution in said receptacle or plurality of receptacles, and the matrix solution and the standard solution are not in substantial admixture. When the device is ready for use, the device or receptacles therein, are warmed to melt the frozen matrix solution and the standard solution, and the liquid test sample is added to the one or more receptacles containing the matrix solution and the standard solution. Upon a certain incubation period, the amount of the analyte is determined using an analyte detection device, for example a mass spectrometer, and the amount of the assayed analyte in the test liquid sample is calculated using the same analyte standards in the standard solution, for example, (only as a non-limiting example, which could apply to any drug or analyte exemplified herein) the amount of cocaine in the test liquid sample (blood, urine or a saliva sample) obtained from a subject, for example, a human subject, is determined using the cocaine standards in the cocaine calibration standards and/or the cocaine internal standards (CS+IS), wherein the cocaine in the cocaine standards are isotopic variants of the cocaine analyte assayed in the test liquid sample.

One embodiment of the invention is a system for the detection of the concentration of cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl, glucuronide conjugated drugs, methylphenedate, 6-MAM or a 6-MAM metabolite in a human liquid sample, the system comprising:
a) a receptacle or plurality of receptacles, each receptacle configured to hold a human sample wherein each receptacle independently comprises: i) a matrix solution comprising at least one of urine, blood, saliva, a beta-glucuronidase solution comprising an enzymatic reaction buffer, and ii) a standard analyte solution comprising a standard analyte solution selected from the group consisting of: a calibration standard, a quality control standard, a process control, an internal standard, and a high temperature melting solvent; wherein the matrix solution is separated from the standard analyte solution. Upon commencement of the assay procedure, the device and its receptacles for example, wells of a multi-well plate are warmed to permit melting and mixing of the standard analyte solution and matrix solution. In some of the receptacles, mixing of the standard analyte solution and matrix solution which are previously, concurrently or subsequently added to the human liquid sample in the receptacle or plurality of receptacles;
b) wherein the analyte to be detected and measured in the human liquid test sample is cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl, glucuronide conjugated drug, methylphenedate, 6-MAM or a 6-MAM metabolite. The standard solution containing isotopic variants of the analyte to be measured and the presence of the analyte in the test wells or receptacles are determined using an analyte detection device that is able to measure and distinguish between the analyte standards and the analyte present in the human liquid test sample.

Another embodiment of the invention is a device and system for the detection of heroin or 6-MAM in a human liquid sample, the system comprising: a) a receptacle or plurality of receptacles, each receptacle configured to hold a human sample wherein each receptacle independently comprises: i) a matrix solution comprising urine, blood, saliva, beta-glucuronidase solution comprising an enzyme buffer or any combination thereof, and ii) a standard solution comprising a 6-MAM standard selected from the group consisting of: a calibration standard, a quality control standard, a process control, an internal standard, and a high temperature melting solvent; wherein the matrix solution is separated from the standard solution in said receptacle or plurality of receptacles, and the matrix solution and the standard solution are not in contact and not in substantial admixture in said receptacle or plurality of receptacles; b) a 6-MAM detection device operable to quantify the amount of 6-MAM, a 6-MAM metabolite or other glucuronide standards in the human liquid sample relative to the amount of at least one 6-MAM standard in a receptacle containing the human liquid sample; wherein the amount of 6-MAM, 6-MAM metabolite, or other glucuronide standards thereof in the human liquid sample is detected with the 6-MAM detection device after the standard solution and the matrix solution have mixed with the human liquid sample in the receptacle or plurality of receptacles.

In preferred embodiments, the standard solution comprises 0.1 ng/ml to about 1 mg/ml of a 6-MAM and a high temperature solvent. The high temperature solvent comprises DMSO or tert-butyl alcohol. The standard solution and the matrix solution are both frozen and separated in the receptacle or plurality of receptacles containing both the matrix solution and standard solution. In a related embodiments for detection of 6-MAM, 6-MAM metabolite, the matrix solution is a solution containing beta-glucuronidase solution comprising an enzyme reaction buffer, and wherein the beta-glucuronidase solution and the standard solution are frozen and separated in the receptacle and are mixed by heating the solutions at the time (or within 0-120 minutes) of assaying the human liquid test sample.

The ability to easily configure the devices of the present disclosure allows for customization according to the particular end user's requirements. The number of tests that may be performed with each device will vary depending upon the number of wells or vials that contain quality control standards, calibration standards, process controls, or are left blank. The standards and the number of wells or vials comprising the standards may vary depending on the particular assays to be performed.

The methods of the present disclosure do not include steps of preparing or adding the calibration, quality control, process controls, or internal standards, to the receptacles of the device, for example, wells or vials of the device by the end user. The presently disclosed devices are manufactured to contain precisely controlled amounts of a matrix solution, for example, a beta-glucuronidase solution comprising an enzymatic reaction buffer and calibration standards, quality control standards, process controls, and internal standards in form as appropriate for the quantitative analysis of a plurality of analytes of interest in a liquid test sample. Preferably the liquid test sample is urine or blood.

In one embodiment, the device of the present invention comprises one or more vials as shown in FIGS. 6A, 6B, and 6C as in a vial tray containing multipole vials, or a vial array contained within a holder capable of holding 1 to 256 vials configured as a single device. In this illustrative embodiment, the device comprises a one or more receptacle 10 which can include a tube or vial, wherein the tube or vial can hold a volume of about 100 μL to about 100 mL. In the illustrative FIGS. 6A-6C noted herein, the vial is a 2.0 mL capacity plastic vial, for example as commercially available by Greiner Bio-One 2 mL external threaded cryovial Item No.: 126263, Greiner Bio-One Monroe, North Carolina, USA. The receptacle (vial) 10 comprises a lid 6 for closure of the receptacle. During manufacture, a matrix 2, for example, blood, saliva or urine is inserted into the bottom of the vial 10 by any means of liquid dispensing, for example, pipetting, liquid injection, or any form of automated liquid dispensing known in the field. The vial 10 is then fitted with a capsule 12 that is preferably degradable in an aqueous solution. As shown in FIG. 6A, the capsule 12 comprises the standard analyte solution 4a which is placed at the bottom of the capsule 12, the amount of standard analyte solution present in the standard analyte solution 4a is made with certain precision, not to exceed plus or minus 10-20% of the stated amount described for the standard analyte solution for example. Then during manufacture of the device, the capsule 12 with the standard analyte solution 4a is inserted into the vial 10 and is held stationary without any further input as the diameter of the capsule 12 is matched to fit snuggly within the inner surface 3 of the vial 10 as shown in FIGS. 6B and 6C. Alternatively, in one illustrative embodiment, capsule 12 in the form of a full capsule and not as shown as a 12 capsule, containing the standard analyte solution 4b either in frozen form or as a dried layer 4b may be placed inside vial 10. Once the capsule 12 is placed within the vial 10, the bottom of the capsule 26 creates a space 30 between the bottom of the capsule 26 and the top of the matrix 22. Space 30 may be any suitable fluid that serves to separate capsule 12 and matrix 2. In some embodiments, space 30 is air or an inert gas for example, nitrogen. In some embodiments, space 30 is an inert fluid or liquid, for example, an oil, a wax, an emulsion, a solution which does not dissolve capsule 12. Space 30 can be any volume within vial 10 such that when the matrix is liquified, the capsule 12 and the matrix 2 can come into contact and permit dissolution of capsule 12. In some embodiments, space 30 is at least 10% of the volume of vial 10, preferably at least 15%, or at least 20%, or at least 25%, or at least 30% of the volume of vial 10. In some embodiments, there is no space between capsule 12 and vial 10 and any other contents in vial 10. Once inserted, the vial 10 containing capsule 12 and matrix 2 the vial 10 can be capped with lid 6 and the assembled vial 10 shown in 1a can be frozen at temperatures ranging from −4° C. to −80° C. and stored in a vial tray (not shown).

In an alternate, but related embodiment, the device of the present invention comprises one or more vials as shown in FIGS. 7A, 7B, and 7C. In this illustrative embodiment, the device comprises one or more receptacle 10 which can include a tube or vial, wherein the tube or vial can hold a volume of about 100 μL to about 100 mL. In the illustrative FIGS. 7A-7C noted herein, the vial is a 2.0 mL capacity vial, for example as commercially available by Greiner Bio-One 2 mL external threaded cryovial Item No.: 126263, Greiner Bio-One Monroe, North Carolina, USA. The receptacle (vial) 10 comprises a lid 6 for closure of the receptacle. During manufacture, a matrix 2, for example, blood, saliva or urine, is introduced into the bottom of the vial 10, by any means of liquid dispensing, for example, pipetting, injection or any form of automated liquid dispensing known in the field. The vial 10 is then fitted with a capsule 12 that is preferably, degradable in an aqueous solution. As shown in FIG. 7A, the capsule 12 comprises the standard analyte solution 4b which is coated or otherwise adhered to the inner surface of the capsule forming a standard analyte solution layer 4b. The coated layer 4b need not cover the entire inner luminal surface of capsule 12, but the amount of standard analyte solution present in the standard analyte solution layer 4b is made with certain precision, not to exceed plus or minus 10-20% of the stated amount described for the standard analyte solution standard for example. Then during manufacture of the device, the capsule 12 with the standard analyte solution later 4b is inserted into the vial 10 and is held stationary without any further input as the diameter of the capsule 12 is matched to fit snuggly within the inner surface 3 of the vial 10 as shown in FIGS. 7B and 7C. Once the capsule 12 is placed within the vial 10, the bottom of the capsule 26 creates a space 30 between the bottom of the capsule 26 and the top of the matrix 22. Space 30 may be any suitable fluid that serves to separate capsule 12 and matrix 2. In some embodiments, space 30 is air or an inert gas for example, nitrogen. In some embodiments, space 30 is an inert fluid or liquid, for example, an oil, a wax, an emulsion, a solution which does not dissolve capsule 12. Space 30 can be any volume within vial 10 such that when the matrix is liquified, the capsule 12 and the matrix 2 can come into contact and permit dissolution of capsule 12. In some embodiments, space 30 is at least 10% of the volume of vial 10, preferably at least 15%, or at least 20%, or at least 25%, or at least 30% of the volume of vial 10. Once inserted, the vial 10 containing capsule 12 and matrix 2 the vial 10 can be capped with lid 6 and the assembled vial 10 as shown in assembly 1b can be frozen at temperatures ranging from −4° C. to −80° C. and stored in a vial tray (not shown).

In the above capsule related embodiments, the capsule 12 fitted into vial 10 can be made of any material that will dissolve and/or degrade in an aqueous solution. Aqueous solutions comprises matrix 2 solutions that will come into contact with capsule 12, for example, blood, urine and saliva. Capsule 12 can be made of hard or soft gelatin compositions, derived from bovine, porcine or plant sources or combinations thereof. Other materials suitable for making aqueous solution dissolving capsules, whether hard or soft may include: plant based gelling excipients, such as starches, natural or synthetic cellulose materials, for example, chemical modified cellulose like hydroxypropyl methylcellulose (HPMC), carrageenan, pullulan, etc. In one embodiment, capsule 12 is a_

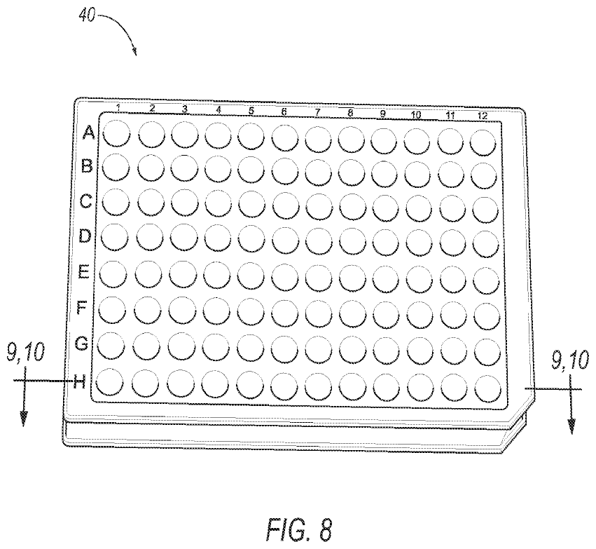

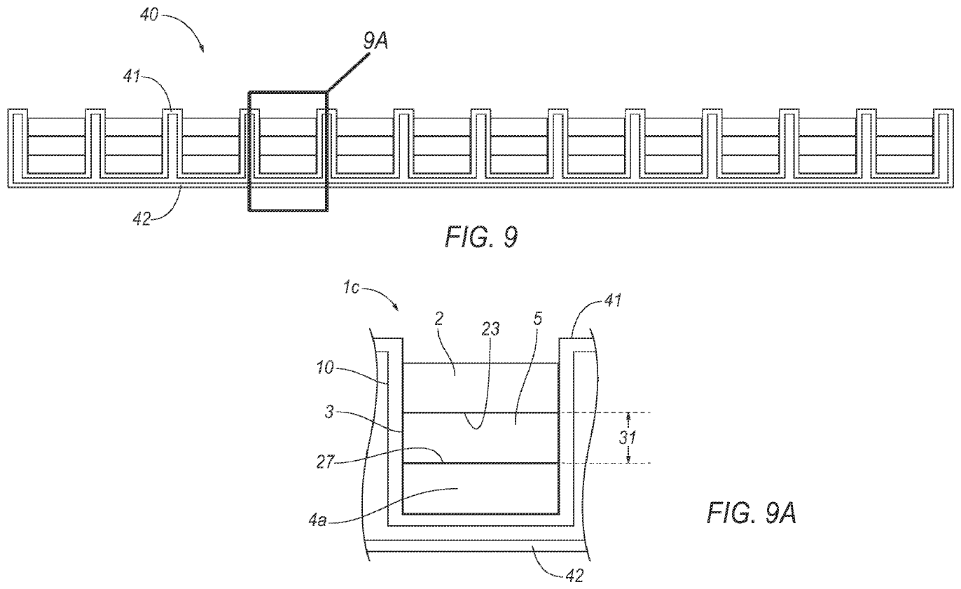

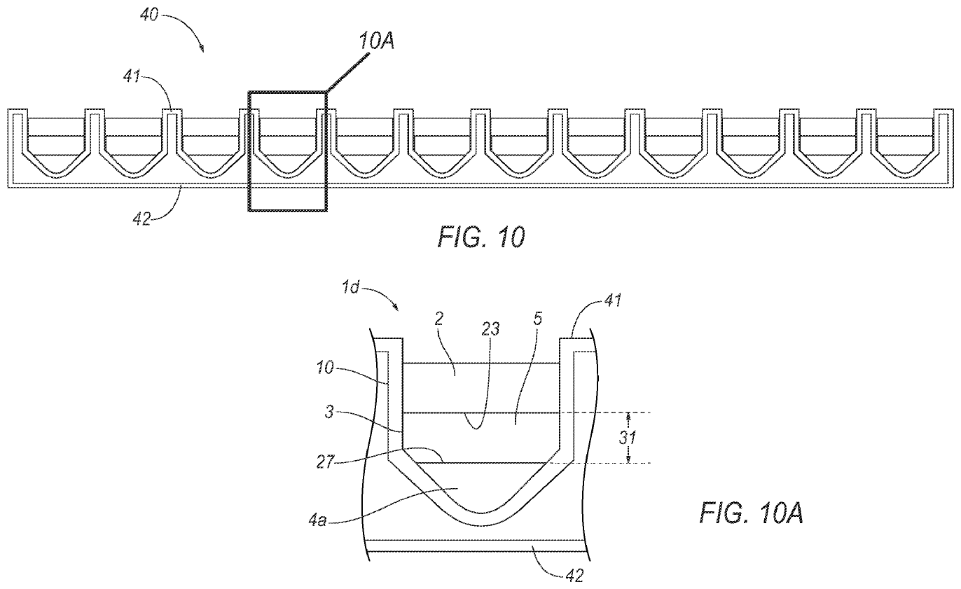

In another embodiment, the device comprises a multi-well plate as shown in FIGS. 8, 9, 9A, 10 and 10A. An illustrative example of an exploded cross-sectional view of FIG. 8 is provided in FIGS. 9, 9A, 10 and 10A in accordance with the several embodiments described herein. FIG. 8 provides a representative device of the present disclosure with a plurality of wells (receptacles) in which to provide the materials of the present disclosure. In FIG. 9, a multi-well plate 40 is shown cross-sectioned as shown in FIG. 8. The microplate 40 contains a plurality of wells 9A and a top panel 41 of multi-well plate 40 and a bottom panel 42 of multi-well plate 40. The well 9A is in an exploded cross-section view shown in FIG. 9A. The device of the present invention can comprise a plurality of wells 9A wherein each well 9A can contain a receptacle 10 defined by a well cavity, being the inner surface 3 of the receptacle 10 in which the well cavity comprises a standard analyte solution 4a having a top surface of bottom fill standard analyte solution upper surface 27. The standard analyte solution 4a can comprise any standard analyte solution to be assayed and described herein. During manufacture, standard analyte solution 4a is introduced into the bottom of the receptacle 10, by any means of liquid dispensing, for example, pipetting, injection or any form of automated liquid dispensing known in the field. The multi-well plate well assembly 1c as shown as a well also comprises a matrix 2. The matrix 2 can comprise blood, saliva or urine, and may be herein generically described as a biological matrix or biological blank matrix, wherein the matrix 2 does not contain the standard analyte solution 4a sought to be tested. Once frozen, standard analyte solution 4a and matrix 2, i.e. between surfaces 27 and 23 define a space 31. Space 31 comprises a barrier 5, which can comprise any suitable fluid that serves to separate standard analyte solution 4a and matrix 2. In some embodiments, barrier 5 is air or an inert gas for example, nitrogen. In some embodiments, barrier 5, can include an inert fluid or liquid, for example, an oil, a wax, an emulsion, a solution which does not permit the admixture of standard analyte solution 4a and matrix 2. In some embodiments, barrier 5 can also comprise gelatin or a plant polysaccharide such as carrageenan, starch or cellulose material, or combinations thereof. Space 31 can be any volume of barrier 5 within receptacle 10 such that when the matrix and/or the standard analyte solution are liquified, the standard analyte solution 4a and the matrix 2 can come into contact and permit mixture within receptacle 10. In some embodiments, space 31 is at least 10% of the volume of receptacle 10, preferably at least 15%, or at least 20%, or at least 25%, or at least 30% of the volume of receptacle 10. In various embodiments, assembly 1c and 1d comprise the receptacle 10 is a well of a multi-well plate, wherein the receptacle 10 can be flat bottomed as shown in FIG. 9A, or round bottomed as shown in FIG. 10A. In several embodiments, the multi-well plate 40 can comprise a lid 6, which rests on the top panel 41 of the multi-well plate 40. The multi-well plate 40 can be constructed or made of any durable solid material, ranging from plastics commonly used in the manufacture of such multi-well plates, for example, polystyrene or polypropylene, but could also include other materials such as ceramics, quartz, glass and other solid materials. The wells of the multi-well assay plate can accommodate any volume commonly employed in the field, for example, ranging from about 5 μL to about 50 mL, and any integer in-between, for example, in a 96 well multi-well assay plate, the internal volume of each receptacle 10 is about 360 μL.

In several embodiments, with reference to FIGS. 9, 9A, 10 and 10A, the relative position of the standard analyte solution 4a and matrix 2 can be inverted, such that layer 2 is the standard analyte solution and layer 4a is the matrix. As used above with reference to FIGS. 6A-6C, 7A-7C, 8, 9, 9A, 10 and 10A, standard analyte solution 4a also includes and refers to analyte standards, including standards used in the present disclosure, for example, CS: calibration standard, QC: quality control standard and IS: internal standard.

### Use of the Devices or Systems for Detection of the Present Disclosure

The devices or systems for detection of the presently disclosed invention simplify the task of preparing test samples for quantitative analysis for the end user of the devices. The device or systems for detection are precisely manufactured to yield consistent results and to reduce the error that can accompany sample preparation.

The device or systems for detection of the presently disclosed invention have receptacles to simplify the task of preparing test samples for quantitative analysis for the end user of the systems for detection. The receptacles are precisely manufactured to yield consistent results and to reduce the error that can accompany sample preparation.

The end user of a device of the present disclosure will provide a test liquid sample to determine the presence and/or quantity of one or more analytes in a biological test sample, for example, a sample of blood, urine, saliva, ascites fluid, cerebrospinal fluid. Preferably the biological test sample is urine, blood, saliva. After preparation of the test sample using an isolation, concentration or purification step, the test sample can be tested directly in some applications but may also be further purified or extracted prior to analysis by any suitable method known in the art such as liquid-liquid extraction, liquid phase extraction, solid phase extraction, supported liquid extraction and high-performance liquid chromatography (HPLC).

Where the biological test sample is urine, blood or saliva or purified urine, blood or saliva it can be analyzed for the amount of an analyte such as cocaine, heroin, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, Fentanyl, glucuronide conjugated drugs, methylphenedate, 6-MAM or a 6-MAM metabolitein the urine, blood or saliva. In particular methods, the analyte is 6-MAM. In general, the higher the level of 6-MAM that is detected by the quantitative analysis of the urine correlates with a higher intake of heroin by the human subject.

This analysis can be performed by any suitable method, such methods are well known in the art, for example gas chromatography (GC), quantitative mass spectrometry tandem mass spectroscopy (MS/MS), liquid chromatography-electrospray tandem mass spectrometry (LC-MS/MS), or liquid chromatography-electrospray time-of-flight mass spectrometry. In other embodiments, analysis of the extracted test sample can be performed by any quantitative analytical method, for example, a mass spectrometric method, an electrophoretic method, NMR, a chromatographic method or a combination thereof. In a further embodiment, the mass spectrometric method is LC-MS and LC-MS/MS. In some embodiments, the LC-MS/MS can be performed using LC-Orbitrap, LC-FTMS, LC-LTQ, MALDI-MS including but not limited to MALDI-TOF, MALDI-TOF/TOF, MALDI-qTOF, and MALDI-QIT. Preferably, the mass spectrometric method is a quantitative using liquid chromatography-electrospray tandem mass spectrometry (LC-MS/MS) with optimized conditions. Other preferred techniques are gas chromatography mass spectrometry (GC-MS) or liquid chromatography mass spectrometry (LC-MS).

Example 1 provides an example of quantitative analysis of multiple drugs in urine by LC-MS/MS.

### Kits

The presently disclosed kits may be used to quantify analytes in a liquid test sample. Preferably the test sample is a urine sample. One embodiment of a kit of the present disclosure includes a kit comprising: a device of the present disclosure and a detailed written description of the specifications of the device and instructions for using the device to perform the chemical analysis and quantification of one or more analytes. In yet another embodiment the kit comprises a device wherein the device is a plurality of vials according to the invention and a detailed written description of the specifications of the device. In another embodiment the kit comprises a device wherein the device is a multi-well plate according to the invention and a detailed written description of the specifications of the device wherein the detailed written description provides the precise amounts of the components in each well or vial of the device. In still another embodiment the kit comprises a device wherein the device is plurality of vials or tubes according to the invention and a detailed written description of the specifications of the device wherein the detailed written description provides the precise amounts of the components in each well or vial of the device.

In some embodiments, the kits of the present disclosure comprise a single device or a plurality of devices as described herein. In some embodiments, the kit includes a device, for example, a 96-well multi-well plate. In another embodiment of the kit, the device is a 384 well multi-well plate. In another embodiment of the kit, the device is a 1024 well multi-well plate. In another embodiment of the kit, the device is a 1536 well multi-well plate. In another embodiment of the kit, the device is having from 20 to 300 vials. In some embodiments the device has from about 1 to about 50, 100, 150, 200, or about 300 vials, the vials optionally configured in a tray or an array.

In another embodiment the kit comprises a supported liquid extraction device wherein the supported liquid extraction device is an Isolute® SLE⁺ 96 well plate or Isolute® SLE⁺ column, Biotage, Charlotte, NC.

In another embodiment the kit comprises a liquid-liquid extraction component wherein the liquid-liquid extraction component consists of one or multiple solutions that are used for sample clean-up. These solutions may include but are not limited to aqueous buffer solutions consisting of 1) ammonium formate, ammonium hydroxide or other bases, 2) organic solutions containing ethyl acetate with or without co-solvents, and 3) aqueous solutions with miscible organic co-solvents such as acetonitrile or methanol.

In any of the kits of the invention, the kit may further comprise standard operating procedures for measuring specific analytes in human urine or blood, or saliva wherein the procedures are customized to meet specific end user validation requirements.

### EXAMPLES

The following examples are for illustrative purposes only and are not intended to limit the scope of the present disclosure. The devices of the present disclosure are highly customizable so as to meet the requirement of a variety of end users.

### Example 1: Quantitative Analysis of Multiple Drugs in Urine by LC-MS/MS

Test samples (urine) are obtained from test subjects and are frozen prior to use. To a plate configured according to Table 1, a sample is added to the wells identified as CS+IS+Glc, QC+IS+Glc, blank+Glc, or blank. Except for the blank sample, each well contains 9000 units of β-glucuronidase enzyme in 150 μL of buffer solution. The blank sample comprises 50 μl of a urine sample that does not contain any of the analytes to be quantified, 150 μl of 10 mM ammonium acetate and 0.5% acetic acid (pH 4.6) (or other appropriate buffer) with 9000 units of β-glucuronidase enzyme. 50 μl of test samples (urine) in duplicate are added to wells identified as IS along with 150 μl of enzyme/buffer solution; the contents of the wells are simultaneously incubated at 37° C. mixed for 30 min in an orbital shaker. After mixing, 500 μl of 0.5 molar ammonium hydroxide buffer is added to all wells, and the contents of the wells are processed in parallel by loading the contents into corresponding wells of a supported liquid extraction plate (e.g., a Isolute® SLE plate, Biotage Charlotte, NC) or subjecting to a liquid extraction technique. The analytes are extracted or eluted from SLE, dried, and reconstituted in methanol (100 μl) The processed samples are then analyzed by positive electrospray ionization LC-MS/MS to obtain the level of 6-MAM and morphine in the sample. The higher the level of 6-MAM generally correlates with a greater intake of heroin by the subject.

The method described in this example is for exemplary purposes. Other methods of quantification are described in this application and are well known to one skilled in the art, and are suitable for use in conjunction with the devices of the present disclosure.

TABLE 1
Table 1: Customized Device for the Quantification of Multiple Drugs in Urine

| Ref. | 1 | 2 | 3 | 4 | 5 | 6 | 7 | 8 | 9 | 10 | 11 | 12 |
|---|---|---|---|---|---|---|---|---|---|---|---|---|
| A | CS + | QC + | IS + | IS + | IS + | IS + | IS + | IS + | IS + | IS + | QC + | Blank + |
| | IS + | IS + | Glc | Glc | Glc | Glc | Glc | Glc | Glc | Glc | IS + | Glc |
| | Glc | Glc | | | | | | | | | Glc | |
| B | CS + | QC + | IS + | IS + | IS + | IS + | IS + | IS + | IS + | IS + | QC + | Blank + |
| | IS + | IS + | Glc | Glc | Glc | Glc | Glc | Glc | Glc | Glc | IS + | Glc |
| | Glc | Glc | | | | | | | | | Glc | |
| C | CS + | QC + | IS + | IS + | IS + | IS + | IS + | IS + | IS + | IS + | QC + | Blank + |
| | IS + | IS + | Glc | Glc | Glc | Glc | Glc | Glc | Glc | Glc | IS + | Glc |
| | Glc | Glc | | | | | | | | | Glc | |
| D | CS + | QC + | IS + | IS + | IS + | IS + | IS + | IS + | IS + | IS + | QC + | Blank + |
| | IS + | IS + | Glc | Glc | Glc | Glc | Glc | Glc | Glc | Glc | IS + | Glc |
| | Glc | Glc | | | | | | | | | Glc | |
| E | CS + | QC + | IS + | IS + | IS + | IS + | IS + | IS + | IS + | IS + | QC + | Blank + |
| | IS + | IS + | Glc | Glc | Glc | Glc | Glc | Glc | Glc | Glc | IS + | Glc |
| | Glc | Glc | | | | | | | | | Glc | |
| F | CS + | QC + | IS + | IS + | IS + | IS + | IS + | IS + | IS + | IS + | QC + | Blank + |
| | IS + | IS + | Glc | Glc | Glc | Glc | Glc | Glc | Glc | Glc | IS + | Glc |
| | Glc | Glc | | | | | | | | | Glc | |
| G | CS + | QC + | IS + | IS + | IS + | IS + | IS + | IS + | IS + | IS + | QC + | Blank + |
| | IS + | IS + | Glc | Glc | Glc | Glc | Glc | Glc | Glc | Glc | IS + | Glc |
| | Glc | Glc | | | | | | | | | Glc | |
| H | CS + | QC + | IS + | IS + | IS + | IS + | IS + | IS + | IS + | IS + | QC + | Blank |
| | IS + | IS + | Glc | Glc | Glc | Glc | Glc | Glc | Glc | Glc | IS + | |
| | Glc | Glc | | | | | | | | | Glc | |

CS: calibration standard
QC: quality control standard
IS: internal standard
Glc: B-Glucuronidase enzyme

TABLE 2
Table 2: Customized Device for the Quantification of Multiple
Drugs in Blood, Urine, Oral Fluid, or other biological matrix.

| Ref. | 1 | 2 | 3 | 4 | 5 | 6 | 7 | 8 | 9 | 10 | 11 | 12 |
|---|---|---|---|---|---|---|---|---|---|---|---|---|
| A | CS + | QC + | IS ++ | IS ++ | IS ++ | IS ++ | IS ++ | IS ++ | IS ++ | IS ++ | QC + | Blank + |
| | IS + | IS + | matrix | matrix | matrix | matrix | matrix | matrix | matrix | matrix | IS + | matrix |
| | matrix | matrix | | | | | | | | | matrix | |
| B | CS + | QC + | IS ++ | IS ++ | IS ++ | IS ++ | IS ++ | IS ++ | IS ++ | IS ++ | QC + | Blank + |
| | IS + | IS + | matrix | matrix | matrix | matrix | matrix | matrix | matrix | matrix | IS + | matrix |
| | matrix | matrix | | | | | | | | | matrix | |
| C | CS + | QC + | IS ++ | IS ++ | IS ++ | IS ++ | IS ++ | IS ++ | IS ++ | IS ++ | QC + | Blank + |
| | IS + | IS + | matrix | matrix | matrix | matrix | matrix | matrix | matrix | matrix | IS + | matrix |
| | matrix | matrix | | | | | | | | | matrix | |
| D | CS + | QC + | IS ++ | IS ++ | IS ++ | IS ++ | IS ++ | IS ++ | IS ++ | IS ++ | QC + | Blank + |
| | IS + | IS + | matrix | matrix | matrix | matrix | matrix | matrix | matrix | matrix | IS + | matrix |
| | matrix | matrix | | | | | | | | | matrix | |
| E | CS + | QC + | IS ++ | IS ++ | IS ++ | IS ++ | IS ++ | IS ++ | IS ++ | IS ++ | QC + | Blank + |
| | IS + | IS + | matrix | matrix | matrix | matrix | matrix | matrix | matrix | matrix | IS + | matrix |
| | matrix | matrix | | | | | | | | | matrix | |
| F | CS + | QC + | IS ++ | IS ++ | IS ++ | IS ++ | IS ++ | IS ++ | IS ++ | IS ++ | QC + | Blank |
| | IS + | IS + | matrix | matrix | matrix | matrix | matrix | matrix | matrix | matrix | IS + | |
| | matrix | matrix | | | | | | | | | matrix | |
| G | CS + | QC + | IS ++ | IS ++ | IS ++ | IS ++ | IS ++ | IS ++ | IS ++ | IS ++ | QC + | CS ++ |
| | IS + | IS + | matrix | matrix | matrix | matrix | matrix | matrix | matrix | matrix | IS + | matrix |
| | matrix | matrix | | | | | | | | | matrix | |
| H | CS + | QC + | IS ++ | IS ++ | IS ++ | IS ++ | IS ++ | IS ++ | IS ++ | IS ++ | QC + | QC ++ |
| | IS + | IS + | matrix | matrix | matrix | matrix | matrix | matrix | matrix | matrix | IS+ | matrix |
| | matrix | matrix | | | | | | | | | matrix | |

CS: calibration standard
QC: quality control standard
IS: internal standard
matrix: blank blood, urine, oral fluid, or other biological material

### Example 2: Manufacturing Process A

A plate as described in Table 1, is created by adding an aqueous solution of B-glucuronidase enzyme in buffer to each well, either as a frozen plug or by subsequently freezing the solution contained in each well. To the aqueous enzyme solutions while in the frozen state, is added the desired mixture of analytes, in the present disclosure the analytes are 6-MAM or morphine, formulated in a high-melting solvent. Preferred high-melting solvents include but are not limited to DMSO and t-butyl alcohol. The analyte solution may be frozen prior to addition, frozen on contact with the chilled enzyme solution, or subsequently frozen after addition to maintain the separated layers.

### Example 3: Manufacturing Process B

A plate as described in Table 1 is created as described in example 2 (manufacturing process A), but by reversing the order of addition of the aqueous and organic phases.

### Example 4: Manufacturing Process C

A plate as described in examples 2 and 3 but with an aqueous or organic layer that is devoid of B-glucuronidase enzyme and analytes to serve as a frozen physical barrier between the enzyme and analyte layers. This barrier layer may include but not be limited to water, DMSO, and t-butyl alcohol.

### Example 5: Manufacturing Process D

A plate as described in Table 1, is created by adding a frozen plug of glucuronidase enzyme in buffer to each well to the desired mixture of analytes, formulated in a high-melting solvent. Preferred high-melting solvents include but are not limited to DMSO and t-butyl alcohol. The frozen plug of enzyme may be formulated with a small rod made of paper, plastic, or other inert material frozen within and extruding from the plug to provide physical separation between the frozen layers. Alternatively, a physical barrier made of paper, plastic, or other inert material may be insert into the well to serve as a physical barrier between the frozen layers.

### Example 6: Manufacturing Process E

A plate as described in Table 1, is created by adding a frozen plug of glucuronidase enzyme in buffer, contained within a cylindrical paper or plastic tube, lodged into to each well in such a way to provide physical separation between the enzyme layer and the analyte layer formulated in a high-melting solvent. Preferred high-melting solvents include but are not limited to DMSO and t-butyl alcohol. The frozen plug of enzyme may be formulated with a channel through the interior of the frozen plug to facilitate insertion into the well.

### Example 7: Manufacturing Process F

A plate as described in Table 1, is created by adding a frozen plug of glucuronidase enzyme in buffer, lodged into to each well in such a way to provide physical separation between the enzyme layer and the analyte layer formulated in a high-melting solvent. Preferred high-melting solvents include but are not limited to DMSO and t-butyl alcohol.

### Example 8: Manufacturing Process G

A plate as described in Table 1, is created by adding a solid carrier material that enzyme may adhere or be absorbed, such as paper, into to each well in such a way to provide physical separation between the enzyme layer and the analyte layer formulated in a high-melting solvent. Preferred high-melting solvents include but are not limited to DMSO and t-butyl alcohol. The enzyme on carrier material may be added directly while in a liquid state, frozen prior to addition to the well, or lyophilized on the carrier prior to addition to each well.

### Example 9: Manufacturing Process H

A plate or an array of tubes or other containers as described in Table 2, is created by adding a biological matrix to each tube or well as described in examples 2-8 (methods A-G).

### Example 10: Manufacturing Process I

A tube or array of tubes may be created by combining a tube with frozen blood with a screw cap seal containing 1 to 100 analytes, frozen within the cap, in a high-melting solvents include but are not limited to DMSO and t-butyl alcohol. In one embodiment, the tube is a 1 mL Thermo Scientific Matrix tube (catalog #NC0685592) with a screw top closure.

Tables 1 and 2 represents an exemplary configuration of wells in a particular embodiment of a device of the present disclosure manufactured for the quantification multiple drugs in a plurality of urine test samples. In particular, the drugs to be analyzed are 6-MAM or morphine. Each well is referred to using the row reference with the column reference. For example, the well in uppermost row and in the left most column is referred to as A1. The plates are manufactured by adding, to the wells of the plate, a beta-glucuronidase solution comprising an enzymatic reaction buffer, the plates are frozen at −80°, to the well of the frozen plate, specific amounts of an organic solution of a calibration standard solution, a quality control standard mix, process control, an internal standard spiking mix or a combination thereof. The procedure for making the device of Example 1 is described below.

A matrix solution is prepared by adding an aqueous solution of 9000 units of B-glucuronidase enzyme in 150 μL of enzymatic reaction buffer is added to each well, except the blank, and the plates are frozen at −80°. The standard solutions are prepared to add to the frozen plates. Quick addition of the DMSO solution of the analytical standards under cold conditions successfully prevented the analytical standards from mixing with the beta-glucuronidase containing matrix solution. After adding the analytical standards, the plates are then stored at −20° C. until needed for use.

The calibration standard mix comprises calibration standards for each analyte to be quantified, in the present disclosure the analytes include 6-MAM and morphine. The calibration standard mix is added in an amount such that the wells comprise amounts of the calibration standards at 0.25, 0.5, 1.25, 2.5, 5.0, 12.5, 25 and 50 ng. Specifically wells A1 and B1 are prepared by adding 50 or 100 μL of a 0.005 μg/mL calibration standard mix to the respective locations; wells C1, D1, and E1 are prepared by adding 25, 50, and 100 μL of a 0.05 μg/mL calibration standard mix to the respective locations; and wells F1, G1, and H1 are prepared by adding 25, 50, and 100 μL of a 0.5 μg/mL calibration standard mix to the respective locations.

The quality control standard mix comprises quality control standards for each analyte to be quantified, in the present disclosure the analytes include one or more standard solutions comprising cocaine, heroin, glucuronide conjugated drugs, methylphenedate, 6-MAM or a 6-MAM metabolite. The quality control mix is added to the wells of the plate such that the wells comprise amounts of the quality control standards at concentrations of 0.25, 0.5, 1.25, 2.5, 5.0, 12.5, 25 and 50 ng. Specifically, wells A2 and B2 are prepared by adding 50 or 100 μL of a 0.005 μg/mL standard solution to the respective locations; wells C2, D2, and E2, are prepared by adding 25, 50, and 100 μL of a 0.05 μg/mL quality control standard mix to the respective locations; and wells F2, G2, and H2 are prepared by adding 25, 50, and 100 μL of a 0.5 μg/mL quality control standard mix to the respective locations. The wells of A11-H11 are loaded in the same manner as corresponding wells A2-H2.

25 μL of internal standard spiking mix is added to all wells of the plate with the exception of column 12. The internal standard spiking mix comprises internal standards at varying concentrations 12.5 to 37.5 ng depending on the particular internal standard.

While in solid state and under cold conditions, 6-MAM analytical standards, formulated in methanol and subsequently diluted with DMSO (final methanol content <10%), were added to the top of this mixture. Quick addition of the DMSO solution under cold conditions successfully prevented the 6-MAM analytical standard from mixing with the beta-glucuronidase (9,000 units) All of the standards are prepared in an organic solvent which may include DMSO, methanol, or t-butyl alcohol.

Suitable reference standard mixes can be obtained from commercial sources for example from Fisher Scientific, Pittsburgh, PA; Sigma-Aldrich, St. Louis, MO; Cayman Chemical, Ann Arbor, MI; Cerilliant, Round Rock, Texas; Cambridge Isotopes, Tewksbury, MA or Lipomed, Cambridge, MA.

### Example 11: Broad Classes of Glucuronides Stabilized in Ready-to-Use Analytical Test Kits Manufactured with Suspended Solid State and Solid State (Direct Enzyme Interface, and Urine Matrix Interface) Technology

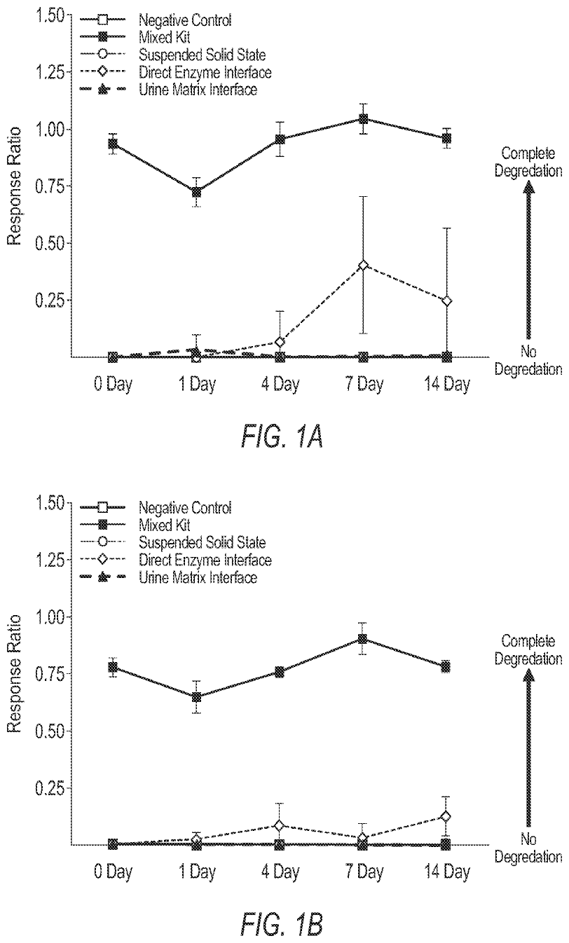

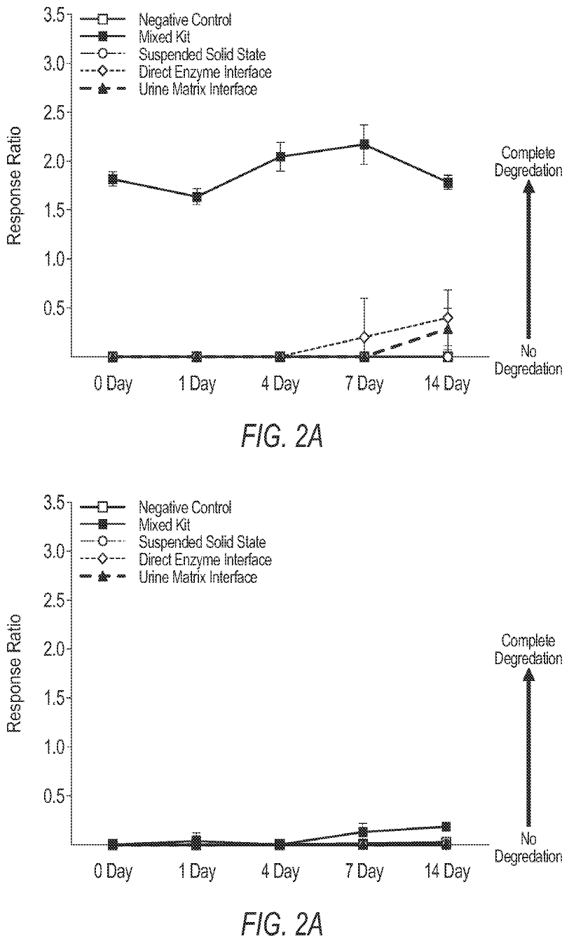

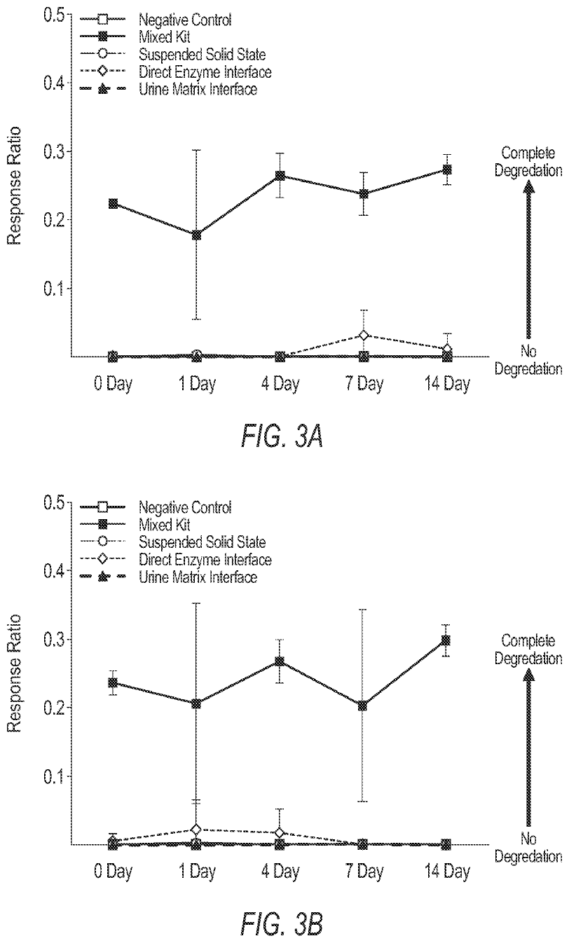

FIGS. 1-4 show that snap freezing process control samples that contain broad classes of glucuronic acid standards does not prevent significant degradation if the manufacturing processes mix either purified recombinant beta-glucuronidase enzyme (KURA) (Panel A for FIGS. 1-4) or beta-glucuronidase isolated from abalone tissue (Panel B for FIGS. 1-4) with the analytical standard. Therefore, novel technology is required to stabilize process control specimens if ready-to-use formats are to be developed for analytical laboratories. Three new technologies (suspended solid state, direct enzyme interface, and urine matrix interface technology) were investigated to determine their utility in ready-to-use analytical test kit designs. While all three technologies afforded significant protection and stabilized the manufacturing process, only the suspended state and urine matrix technologies provided complete stability for 14 days in kits stored at −20° C.

Comparisons with both positive and negative controls show that significant degradation occurs with every class of glucuronic acid standard tested on the day of manufacturing (day 0). The amount of degradation is independent of the source of the beta glucuronidase and remains constant for 14 days when kits are stored at −20° C. This storage temperature was selected because it is mostly commonly utilized by analytical testing laboratories. Degradation is specifically associated with manufacturing and storage because acetonitrile used for sample extraction was added immediately to frozen kits to degrade the beta-glucuronidase enzyme prior to sample processing and where possible (solid-state, direct enzyme interface, and matrix interface samples) the beta-glucuronidase enzyme was removed prior to sample extraction. These measures minimized any degradation that may have occurred during sample processing.

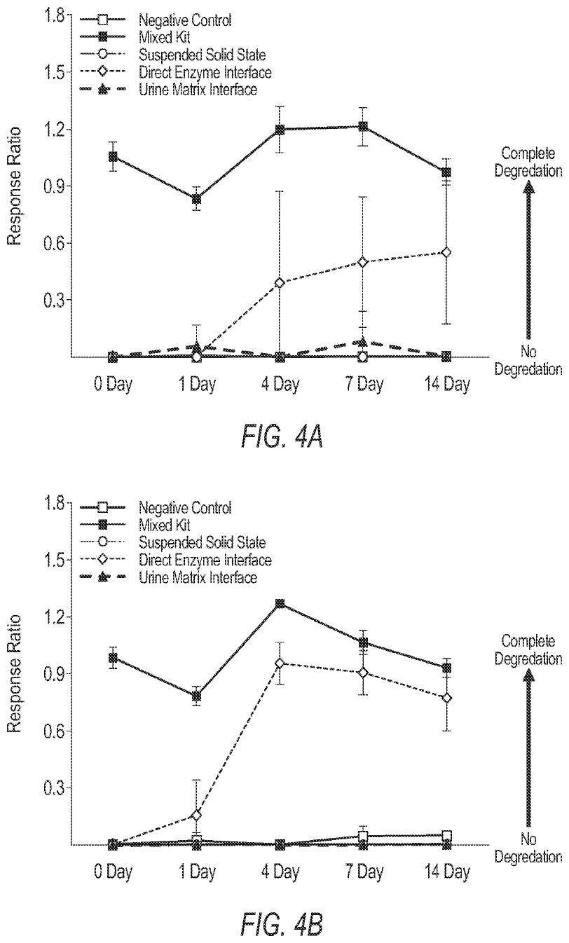

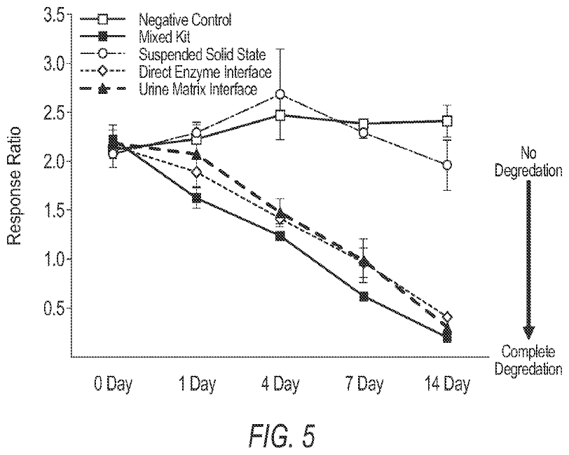

Studies with the lorazepam glucuronic acid standard show that the three technologies offer unique and specific advantages (FIG. 4). The lorazepam glucuronic acid standard was fully protected with both the suspended solid-state technology and urine matrix technology. However, the direct enzyme interface technology only protected this substrate during the manufacturing process and in kits stored for 1 day at −20° C. Significant degradation was detected at day 4, with complete degradation occurring at day 14. Similar results were observed with both the purified recombinant enzyme (KURA) and with the beta glucuronidase isolated from abalone tissue.

### Example 12: Analytical Standards Protected from Non-Specific Matrix Affects in Ready-to-Use Analytical Test Kits Manufactured with Suspended Solid State and Solid State (Direct Enzyme Interface, and Urine Matrix Interface) Technology

Non-specific matrix affects, those not specifically associated with the catalytic activity of beta-glucuronidase, are also known to degrade sensitive analytical standards used in analytical testing laboratories. For example, 6-MAM is prone to hydrolysis and FIG. 6 offers an example of why laboratories often incorporate testing strategies to stabilize and control for 6-MAM degradation. FIG. 6 shows 6-MAM is not stable in analytical test kits manufactured in a ready-to-use format that mixes beta glucuronidase isolated from abalone tissue with the analytical standard. While snap freezing at −20° C. initially stabilizes 6-MAM during the manufacturing process (day 0), significant 6-MAM degradation occurs within 4 days when kits are stored at −20° C. Degradation was time-dependent with complete degradation occurring within 7 to 14 days. Suspended state-technology was the only technology tested that fully protected 6-MAM. Direct enzyme interface and urine matrix interface technologies slowed the rate of degradation but did not prevent 6-MAM degradation. 6-MAM degradation appears to be a non-specific matrix affect since 6-MAM is not a known to be a substrate for beta-glucuronidase, since no degradation occurred during the manufacturing process, and since no 6-MAM degradation occurred in identical analytical test kits manufactured with purified recombinant enzyme (KURA) (data not shown).

### Example 13: Analytical Standards Protected from Non-Specific Matrix Affects in Ready-to-Use Analytical Test Kits Manufactured with Suspended Solid State and Solid State (Blood Matrix Interface) Technology

Matrix affects may occur in blood to degrade sensitive analytical standards used in analytical testing laboratories. For example, heroin degrades to 6-MAM and morphine in blood. Another example is the degradation of cocaine into benzoylecgonine in blood samples. Suspended state-technology as described in the present disclosure by separating the matrix solution from the standard solution as described in the present disclosure and in particular manufacturing process A-I (examples 2-10) offers protection of these examples and other analytes.

### Example 14: Manufacturing Process J

A tube, vial, well or array of tubes or vials or wells may be created by inserting half of an empty pill capsule shell into a tube with frozen matrix. The capsule shell may be previously or subsequently filled with a containing 1 to 100 analytes at single or various concentrations, in one or more high-melting solvents, which may include, but are not limited to, DMSO and t-butyl alcohol for storage in frozen state. In one embodiment, the tube is a 2 mL Greiner Bio-One tube (item #126261) with a screw top closure. As shown in FIG. 6A-6C, a device may comprise one or more of vials as shown in FIGS. 6A-6C, wherein the standard analyte solution (for example analyte standards) is placed within an aqueous solution dissolving capsule, and a matrix solution is placed in the vial such that the manufactured device comprises one or more receptacles (vials), wherein the standard analyte solution and standards are not in admixture with the matrix material. The matrix material is contacted with the standard analyte solution, for example, analyte standards once the test sample is processed and analyzed in parallel. In various embodiments, the matrix material can be a blank matrix material comprising any one of blood, urine or saliva, optionally with a beta-glucoronidase enzyme and enzyme buffer in the matrix material.

### Example 15: Manufacturing Process K

A tube, vial, well or array of tubes or vials or wells may be created by inserting half of an empty pill capsule shell into a tube with frozen matrix. The capsule shell may be previously or subsequently filled with a containing 1 to 100 analytes in a volatile solvent including but are not limited to methanal, ethanol, acetone, hexane, acetonitrile, or ethyl acetate, then evaporated to a dry residue within an empty pill capsule shell as exemplified here in FIGS. 7A, 7B and 7C. As shown in FIG. 6A-6C, a device may comprise one or more of vials as shown in FIGS. 7A, 7B and 7C, wherein the standard analyte solution (for example analyte standards) is placed within an aqueous solution dissolving capsule, and a matrix solution is placed in the vial such that the manufactured device comprises one or more receptacles (vials), wherein the standard analyte solution and standards are not in admixture with the matrix material. The matrix material is contacted with the standard analyte solution, for example, analyte standards once the test sample is processed and analyzed in parallel. In various embodiments, the matrix material can be a blank matrix material comprising any one of blood, urine or saliva, optionally with a beta-glucoronidase enzyme and enzyme buffer in the matrix material.

### Example 16. Heroin Suspended State Device

A 48-well plate from Innovative Labs (48 Rectangular Well U-Bottom 42.60 mm Height, 4.6 mL capacity) was formulated with various combinations of heroin (purchased from Lipomed, item #M-29-FB-1LA, lot #29.3B42.1L2) in DMSO (purchased from Fisher chemical, item 3 D128-1), human blood (blank whole blood purchased from UTAK, and 0.5M NH4OH detailed in the plate layout diagram below.

Wells A1, A2, and A3 designated as “drug only” contain 50 μL of heroin in DMSO at a concentration of 1000 ng/mL. The materials in these wells were store in frozen state before use.

Wells B1, B2, and B3 designated as “drug+blood NH4OH” contain 50 μL of heroin in DMSO at a concentration of 1000 ng/mL mixed with 100 uL 0.5M NH4OH. The materials in this well were stored in frozen state before use.

Wells C1, C2, and C3 designated as “drug+blood popsicle” contain 50 μL of heroin in DMSO at a concentration of 1000 ng/mL combined with 100 μL of frozen blood on a small wooden stick (toothpick), suspended above the frozen DMSO solution by the stick (see example 5: manufacturing process D). The materials in these wells were store in frozen state before use.

Wells D1, D2, and D3 designated as “drug+NH4OH popsicle” contain 50 μL of heroin in DMSO at a concentration of 1000 ng/mL combined with 100 μL of frozen 0.5M NH4OH on a small wooden stick (toothpick), suspended above the frozen DMSO solution by the stick (see example 5: manufacturing process D). The materials in these wells were store in frozen state before use.

Wells E1, E2, and E3 designated as “drug+blood/NH4OH popsicle” contain 50 μL of heroin in DMSO at a concentration of 1000 ng/mL combined with 100 μL of frozen blood and 100 μL of 0.5M NH4OH on a small wooden stick (toothpick), suspended above the frozen DMSO solution by the stick (see example 5: manufacturing process D). The materials in this well were stored in frozen state before use.

Wells F1, F2, and F3 designated as “DMSO (no drug)+blood popsicle” contain 50 μL of DMSO combined with 100 μL of frozen blood on a small wooden stick (toothpick), suspended above the frozen DMSO solution by the stick (see example 5: manufacturing process D). The materials in these wells were store in frozen state before use.

Wells G1, G2, and G3 designated as “DMSO (no drug)+NH4OH popsicle” contain 50 μL of DMSO combined with 100 μL of frozen 0.5M NH4OH on a small wooden stick (toothpick), suspended above the frozen DMSO solution by the stick (see example 5: manufacturing process D). The materials in these wells were store in frozen state before use.

Wells H1, H2, and H3 designated as “DMSO (no drug)+blood/NH4OH popsicle” contain 50 μL of DMSO combined with 100 μL of frozen blood and 100 μL of frozen 0.5M NH4OH on a small wooden stick (toothpick), suspended above the frozen DMSO solution by the stick (see example 5: manufacturing process D). The materials in these wells were store in frozen state before use.

Wells A4, A5, and A6 designated as “DMSO (no drug)” contain 50 μL of DMSO combined with 100 μL of 0.5M NH4OH. The materials in these wells were store in frozen state before use. Wells B4, B5, and B6 designated as “DMSO only (no drug)” contain 50 μL of DMSO. The materials in these wells were store in frozen state before use.

TABLE 3
Plate Layout Diagram for Heroin suspended state study

| | 1 | 2 | 3 | 4 | 5 | 6 |
|---|---|---|---|---|---|---|
| A | drug only | drug only | drug only | DMSO (no drug) + | DMSO (no drug) + | DMSO (no drug) + |
| | | | | NH4OH mixed | NH4OH mixed | NH4OH mixed |
| B | drug + | drug + | drug + | DMSO only (no | DMSO only (no | DMSO only (no |
| | blood/NH4OH | blood/NH4OH | blood/NH4OH | drug) | drug) | drug) |
| | (mixed) | (mixed) | (mixed) | | | |
| C | drug + blood | drug + blood | drug + blood | | | |
| | popsicle | popsicle | popsicle | | | |
| D | drug + NH4OH | drug + NH4OH | drug + NH4OH | | | |
| | popsicle | popsicle | popsicle | | | |
| E | drug + | drug + | drug + | | | |
| | blood/NH4OH | blood/NH4OH | blood/NH4OH | | | |
| | popsicle | popsicle | popsicle | | | |
| F | DMSO (no drug) + | DMSO (no drug) + | DMSO (no drug) + | | | |
| | blood popsicle | blood popsicle | blood popsicle | | | |
| G | DMSO (no drug) + | DMSO (no drug) + | DMSO (no drug) + | | | |
| | NH4OH popsicle | NH4OH popsicle | NH4OH popsicle | | | |
| H | DMSO (no drug) + | DMSO (no drug) + | DMSO (no drug) + | | | |
| | blood/NH4OH | blood/NH4OH | blood/NH4OH | | | |
| | popsicle | popsicle | popsicle | | | |

### Example 17. Glucuronide Product Suspended State Study

The inventors of the present invention decided to test using new Academy Standards Board (ASB) published standards for forensic urine toxicology. This has direct relevance to improving the existing drug testing technology because private, state and federal crime laboratories are seeking new technology to help meet this standard in a more streamline efficient way. First, a new analytical testing procedure was validated to ASB Standards for LC-MS/MS method validations to meet external laboratory accreditation standards (ISO 17025). Further suitability studies were conducted to show that equivalent results can be generated using the new technology.

A 48-well plate from Innovative Labs (48 Rectangular Well U-Bottom 42.60 mm Height, 4.6 mL capacity) was formulated with various combinations of a mixture of glucuronidated drugs purchased from Cerilliant, Round Rock Texas, US (see details in table below), abalone enzyme (item #CST208) from ChemSci technologies, Belvidere IL, USA, Recombinant B-One (item #B-One-100 mL) enzyme from Kura Bioscience, buffer solution made with 5 mL of acetic acid and 7.71 g of ammonium acetate in 995 mL of pure water, and blank urine (item #OH2060) from Golden West Diagnostics.

TABLE 4
Glucoronidated Compositions tested in Assays

| Comp. # | Glucuronidated Drug Standard | Item# | Conc. |
|---|---|---|---|
| 1 | 11-nor-9-carboxy-Δ9-THC glucuronide | T-038 | 0.1 |
| 2 | Hydromorphone 3-beta-D-glucuronide | H-051 | 0.1 |
| 3 | Codeine-6-beta-D-glucuronide | C-087 | 0.1 |
| 4 | Oxazepam glucuronide | O-023 | 0.1 |
| 5 | Buprenorphine-3-beta-D-glucuronide | B-035 | 0.1 |
| 6 | Lorazepam glucuronide | L-021 | 0.1 |
| 7 | Norbuprenorphine glucuronide | N-045 | 0.1 |
| 8 | Amitriptyline N-beta-D-glucuronide | A-128- | 0.1 |
| | | 1ML | |

An assay 96 well microtiter plate (8×12 format) was used for the assay procedures.

Wells A1, B1, C1, and D1 designated as “Kura Neg. control drugs+H₂O” contain the glucuronide drug standards in 50 μL DMSO mixed with 200 mL of water. The materials in this well were stored in frozen state before use.

Wells E1, F1, G1, and H1 designated as “Kura Pos. control drugs+Enz” contain the glucuronide drug standards in 50 μL DMSO mixed with 200 mL of water and 100 mL of Kura enzyme. The materials in this well were stored in frozen state before use.

Wells A2, B2, C2, and D2 designated as “Abl Neg. control drugs+buffer” contain the glucuronide drug standards in 50 μL DMSO mixed with 250 mL of buffer solution. The materials in this well were stored in frozen state before use.

Wells E2, F2, G2, and H2 designated as “Abl Pos. control drugs+Enz” contain the glucuronide drug standards in 50 μL DMSO mixed with 5 KU of abalone enzyme in 250 mL of buffer solution. The materials in this well were stored in frozen state before use.

Wells A3, B3, C3, and D3 designated as “DB pop removed Kura” contain the glucuronide drug standards in 50 μL DMSO and a suspended frozen mixture of 200 mL of water and 100 mL of Kura enzyme (see example 5: manufacturing process D). The materials in this well were stored in frozen state before use and the frozen enzyme and water mixture was removed prior to analysis of the well contents.

Wells E3, F3, G3, and H3 designated as “DB pop removed Abl” contain the glucuronide drug standards in 50 μL DMSO and a suspended frozen mixture of 5 KU of abalone enzyme in 250 mL of buffer (see example 5: manufacturing process D). The materials in this well were stored in frozen state before use and the frozen enzyme and water mixture was removed prior to analysis of the well contents.

Wells A4, B4, C4, and D4 designated as “DB Enz stick up Kura” contain the glucuronide drug standards in 50 μL DMSO and a suspended frozen mixture of 200 mL of water and 100 mL of Kura enzyme (see example 5: manufacturing process D) with the two frozen interfaces in contact with one another. The materials in this well were stored in frozen state before use.

Wells E4, F4, G4, and H4 designated as “DB Enz stick up Abl” contain the glucuronide drug standards in 50 μL DMSO and a suspended frozen mixture of 5 KU of abalone enzyme in 250 mL of buffer (see example 5: manufacturing process D) with the two frozen interfaces in contact with one another. The materials in this well were stored in frozen state before use.

Wells A5, B5, C5, and D5 designated as “DB urine Kura” contain the glucuronide drug standards in 50 μL DMSO and a layer of frozen urine on top. A mixture of 200 mL of water and 100 mL of Kura enzyme (see example 5: manufacturing process D) with the two frozen interfaces in contact with one another. The materials in this well were stored in frozen state before use.

Wells E5, F5, G5, and H5 designated as “DB urine Kura” contain the glucuronide drug standards in 50 μL DMSO and a layer of frozen urine on top. A mixture of 5 KU of abalone enzyme in 250 mL of buffer (see example 5: manufacturing process D) with the two frozen interfaces in contact with one another. The materials in this well were stored in frozen state before use.

TABLE 5
Plate Layout Diagram for Glucuronide product suspended state study

| | | | | | |
|---|---|---|---|---|---|
| 1 | 2 | 3 | 4 | 5 | 6 |

| | | | | | |
|---|---|---|---|---|---|
| A | Kura Neg. Control | Abl Neg. Cont. | DB Pop. removed | DB Enz. stick up | DB Urine Kura |
| | Drugs + H20 | Drugs + buff | Kura | Kura | |
| B | Kura Neg. Control | Abl Neg. Cont. | DB Pop. removed | DB Enz. stick up | DB Urine Kura |
| | Drugs + H20 | Drugs + buff | Kura | Kura | |
| C | Kura Neg. Control | Abl Neg. Cont. | DB Pop. removed | DB Enz. stick up | DB Urine Kura |
| | Drugs + H20 | Drugs + buff | Kura | Kura | |
| D | Kura Neg. Control | Abl Neg. Cont. | DB Pop. removed | DB Enz. stick up | DB Urine Kura |
| | Drugs + H20 | Drugs + buff | Kura | Kura | |
| E | Kura Pos. Control | Abl. Pos. Control | DB Pop. removed | DB Enz. stick up | DB Urine Abalone |
| | Drugs + Enz. | Drugs + Enz. | Abl. | Abl. | |
| F | Kura Pos. Control | Abl. Pos. Control | DB Pop. removed | DB Enz. stick up | DB Urine Abalone |
| | Drugs + Enz. | Drugs + Enz. | Abl. | Abl. | |
| G | Kura Pos. Control | Abl. Pos. Control | DB Pop. removed | DB Enz. stick up | DB Urine Abalone |
| | Drugs + Enz. | Drugs + Enz. | Abl. | Abl. | |
| H | Kura Pos. Control | Abl. Pos. Control | DB Pop. removed | DB Enz. stick up | DB Urine Abalone |
| | Drugs + Enz. | Drugs + Enz. | Abl. | Abl. | |

### Results

The present experimental example was performed to determine if the suspended state technology stabilize common glucuronic acid conjugates and other sensitive analytical standards like 6-MAM in premanufactured test kits. Other similar studies were conducted to optimize the amount of time necessary to hydrolyze typical glucuronides used as process control in standard validated procedures. Analytical kits used for these studies followed standard validated procedures but pre-manufactured in a ‘ready-to-go’ kit format using suspended state technology. Plates were simply taken out of the freezer and set out at ambient temperature.

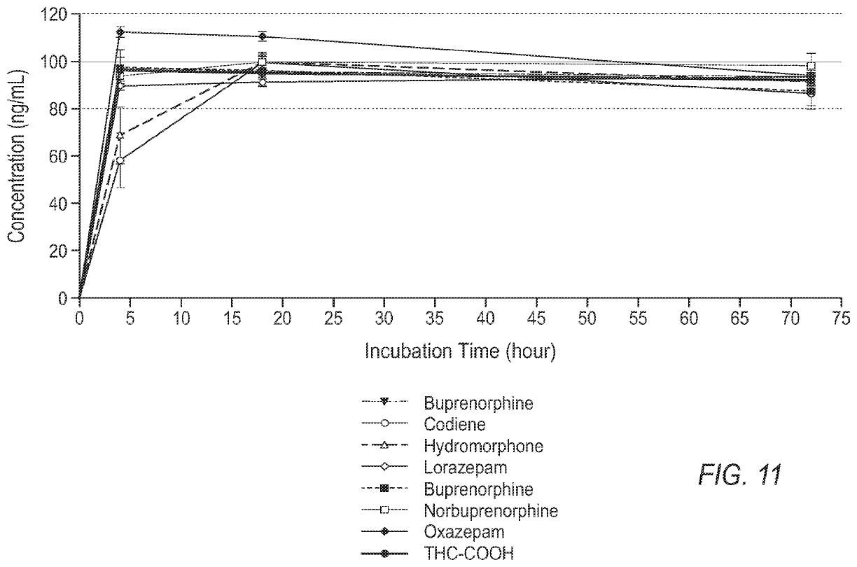

Glucuronides were spiked at a level to yield 100 ng/ml free drug when hydrolysis was complete. ±20% bias is the recommended acceptable range for these studies. Data are presented in FIG. 11 as average bias ±% OCV (N=10). Data show an overnight 18-hour incubation is required to achieve sufficient hydrolysis. Reactions are stable out to 3 days. 3 days was chosen to represent a weekend incubation at ambient temperature.

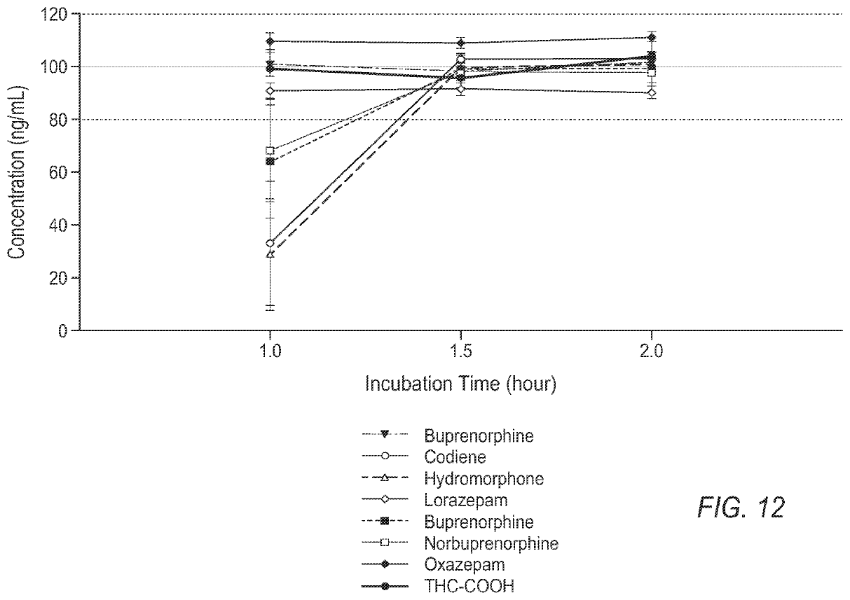

The results shown in FIG. 12 are results from an experiment performed to determine and optimize the amount of time necessary to hydrolyze typical glucuronides used as process control in standard validated procedures. Analytical kits used for these studies followed standard validated procedures but pre-manufactured in a ‘ready-to-go’ kit format using suspended state technology. Plates were simply taken out of the freezer and incubated at 60° C. Glucuronides were spiked at a level to yield 100 ng/ml free drug when hydrolysis was complete. ±20% bias is the recommended acceptable range for these studies. Data are presented as average bias ±% CV (N=10). Data show that 1.5 hrs is required to achieve sufficient hydrolysis and analytes. Reactions are stable for 2 hrs at 60° C. Heating the premanufactured plate decreased the amount of time necessary to release reaction components manufactured in suspended state.

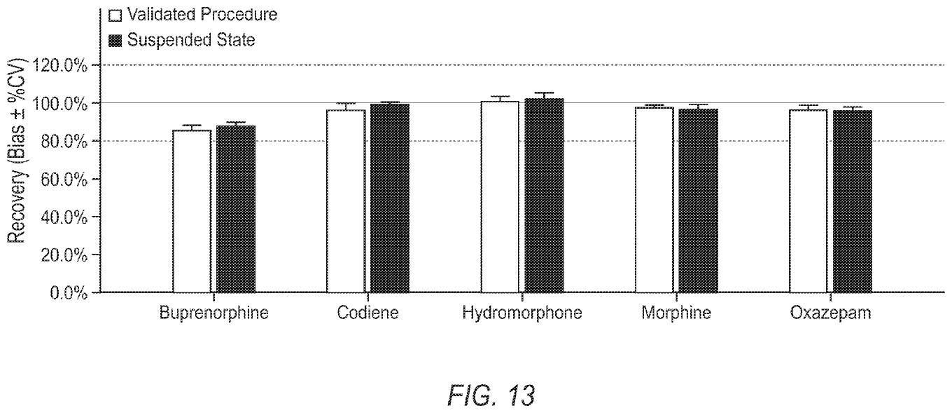

Glucuronic acid conjugates of varying drug classes are often used as process controls in standard validated urine toxicology panels recommended by ASB. Results shown in FIG. 13 are illustrative of a series of experiments comparing the existing ASB compliant validated procedure using the same drug panel run with new suspended state technology. Samples shown in FIG. 13 represent the glucuronides spiked in urine matrix to access the efficacy of the beta glucuronidase enzyme. ‘Ready-to-Go’ suspended state analytical kits of the present invention were incubated at 60° C. for 2 hrs and free drug assayed as a measure of efficacy. These quality control specimens are spiked to deliver 100 ng/ml final free drug concentration and ±20% bias is the recommended acceptable range. Data are presented as average bias ±% CV (N=3-5). Data show equivalency between a standard validated procedure when compared to suspended state technology.

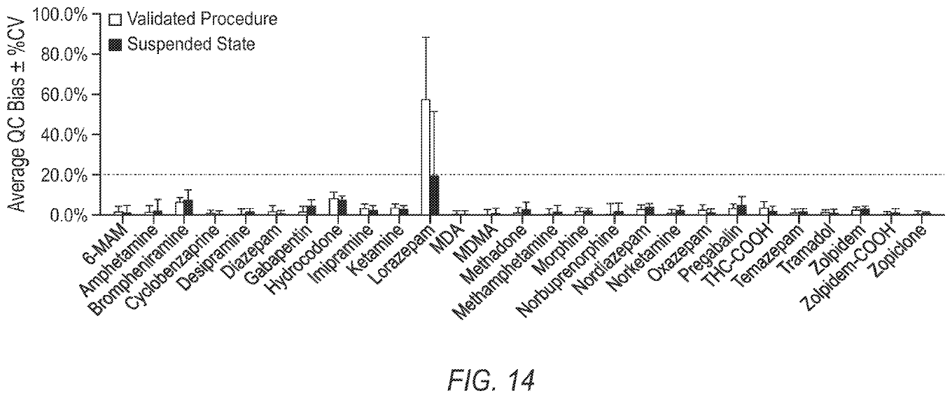

Results shown in FIGS. 14-16 are illustrative of an experiment comparing an ASB compliant validated procedure, with the same drug panel, run with new suspended state technology of the present invention. Samples were spiked with 5 levels of QC material spanning the reportable range. Bias measurements represent average recovery of all levels ±% CV. Data presented in FIGS. 14-16 are represented as average bias ±% CV (N=36-100). All drugs exceed method performance criteria except lorazepam, which failed acceptable criteria in both experimental groups. The internal standard clonazepam-D4 interfered with lorazepam and prevented accurate measurements of this drug. Data shown in FIGS. 14-16 validate the suspended state technology described in the present disclosure as equivalent to standardized, validated urine toxicology methods. The selected drug menu is representative of the ASB recommendations for the forensic testing of urine collected as part of drug facilitated crimes.

### Example 18. ToxBox® Devices of the Present Disclosure

### Experimental Section

### Reagents and Chemicals

The Academy Standards Board (ASB) compliant ToxBox® kit provided by PinPoint Testing, LLC (Little Rock, AR) streamlines sample preparation and testing procedures to allow high-throughput testing capacity. This kit incorporates NIST-traceable, certified reference material for all standards and isotopically-labeled internal standards to control for extraction efficiencies. The kit also includes ISOLUTE® SLE+96-well plates manufactured by Biotage (Charlotte, NC). Ammonium hydroxide, ethyl acetate, and methanol were purchased from Fisher Scientific (Fairlawn, NJ). Deionized water was purified to 18.2 MΩ·cm resistivity using the equivalent of a Millipore laboratory water purification system. Unless otherwise specified, all other chemicals and supplies were provided by Cerilliant (Round Rock, TX), Cayman Chemical Company, Biotage or Golden West (Temecula, CA). Blank pooled human urine void of analytes of interest was used for all studies.

### Equipment

Validation studies used supported liquid extraction (SLE) optimized for 96-wellplate processing. Sample extracts were analyzed using an Agilent 1260 quaternary liquid chromatography system (Santa Clara, CA) coupled to an Agilent 6420 tandem mass spectrometer (LC-MS/MS). Instrument control and data acquisition relied on MassHunter LC/MS Data Acquisition (VER B.08.00). Data analysis was performed using MassHunter Quantitative Analysis (VER B.07.01 SP2).

### Preparation of Analytical Standards and Quality Control Material

Analytes of interest for this study included all the analytes listed (cocaine, heroin, glucuronide conjugated drugs, methylphenedate, 6-MAM, 6-MAM metabolite, opiates, opioids, benzodiazepines, stimulants, barbiturates, cannabinoids, novel psychoactive compounds, and other therapeutic drugs such as Carisoprodol, Phenytoin, Valproic Acid, Meprobamate, Topiramate, Levamisole, Propoxyphene, Pseudoephedrine, Metaxalone, Carbamazepine, Lamotrigine, 10-hydroxycarbamazepine, 7-hydroxy-quetiapine, Citalopram, Duloxetine, Meperidine, Normeperidine, Quetiapine, Trazodone, Amitriptyline, Benzoylecgonine, Clomipramine, Clozapine, Desipramine, Desmethylclomipramine, Desmethyltrimipramine, Dextromethorphan, Diphenhydramine, Doxylamine, EDDP, Fluoxetine, mCPP, Nortriptyline, O-Desmethyltramadol, O-Desmethylvenlafaxine, Phentermine, Tramadol, Trimipramine, Venlafaxine, N-Desmethylclobazam, Bupropion, Buspirone, Desmethyldoxepin, Doxepin, Hydroxyzine, Imipramine, Mirtazapine, N-Desmethylclozapine, Amphetamine, Chlordiazepoxide, Chlorpromazine, Clobazam, Cyclobenzaprine, Diazepam, MDA, MDMA, Methadone, Methamphetamine, Metoprolol, Nordiazepam, Norephedrine, Norketamine, Oxazepam, Paroxetine, Ritalinic Acid, Temazepam, Verapamil, 7-aminoclonazepam, Alprazolam, Amoxapine, Chlorpheniramine, Clonazepam, Cocaine, Codeine, Dihydrocodeine, Etizolam, Haloperidol, Hydrocodone, Ketamine, Lorazepam, Maprotiline, Methylphenidate, Mitragynine, Morphine, Norhydrocodone, Noroxycodone, Oxycodone, Phenazepam, Sertraline, Tapentadol, Zaleplon, Zolpidem, Zopiclone, Brompheniramine, Cocaethylene, 2-Hydroxyethylflurazepam, 6-MAM, 7-aminoflunitrazepam, 9-Hydroxyrisperidone, alpha-Hydroxyalprazolam, alpha-Hydroxymidazolam, Desalkylflurazepam, Estazolam, Flualprazolam, Flubromazolam, Flunitrazepam, Hydromorphone, Midazolam, Olanzapine, Oxymorphone, Phencyclidine, Promethazine, Risperidone, Clonazolam, GHB, Flurazepam, Triazolam, LSD, Norbuprenorphine, Norfentanyl, Acetyl fentanyl, Acetyl Norfentanyl, Buprenorphine, and Fentanyl). Analytical standards of each analyte and second source quality control material used for these studies were provided in the THC ToxBox® kit. Standards, QCs, and internal standards are manufactured in a 96-wellplate format to deliver precise concentrations, as described in package inserts.

Prior to analysis, drug residue in each well is reconstituted in 100 μL of blank pooled human urine to build analytical standards (0.1 ng/mL to 100,000 ng/mL) and second source QCs spanning the linear working range (0.3 ng/mL to 90,000 ng/mL). Internal standards also are premanufactured in each standard and QC well in addition to blank wells for unknown specimen analysis. The final internal standard concentration in 100 μl urine samples ranged from 5 to 5000 ng/mL.

### Extraction of Standards, Quality Control Material, and Specimens

All urine calibration standards, QC material, and unknown samples were processed identically by mixing 100 μl of blank pooled human urine, 100 μl of KURA enzyme, and 200 μl of 18.2 MΩ·cm water in appropriate wells at 900 rpm for 30 min at ambient temperature. Reactions were terminated by adding 100 μl of solution A provided as part of the test kit, and then mixing for another 15 min at 900 at ambient temperature. Samples were extracted by adding 600 μl of Solution B provided as part of the test kit. All samples were mixed by aspirating/dispensing ten times. The aqueous and organic layers separated for 10 min prior to the aqueous wasted being removed and discarded. The remaining organic layer was evaporated to dryness at 60° C. and drug residue reconstituted in 200 μl solution C provided as part of the test kit. All extracts were immediately assayed or stored at 4° C. until analysis.

### Liquid Chromatography Tandem Mass Spectrometry

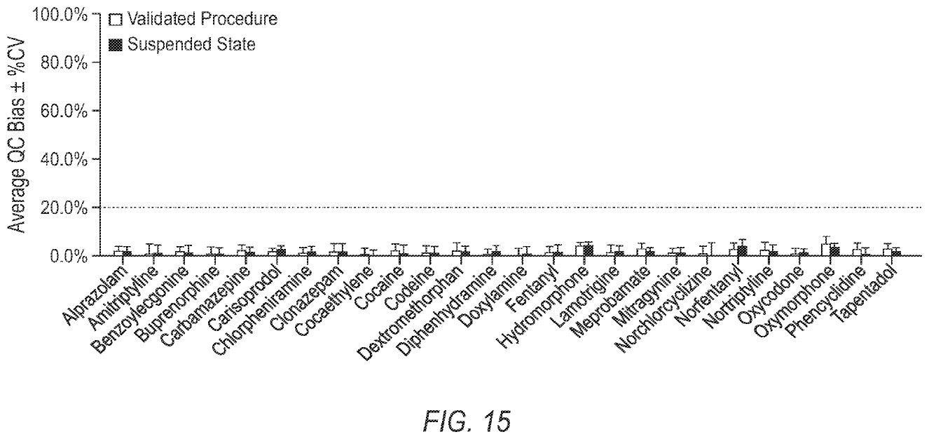

The LC-MS/MS method used for FIGS. 14-15 incorporated 10 μl injections on a 2.6 μm Phenomenex Kinetex 2.6 (50×2.1 mm) LC column heated to 50° C. Analytes were resolved at 0.4 mL/min using mobile phase A (25 mM Ammonium Formate in ultrapure 18.2 MΩ·cm water containing 0.1% formic acid) and mobile phase B (100% methanol).). Initial conditions were 90% mobile phase A/10% mobile phase B.A standard gradient that ramped to 5% mobile phase A/95% mobile phase B was used for all analyses in positive mode. The total run time including column equilibration period between injections was 10 min. Specific mass spectrometer and analyte parameters are specifically optimized for each analyte of interest. In general, two transitions were monitored for each analyte. Ion ratios were matched to those of calibration standards to ensure interfering metabolites and other compounds were resolved. To ensure carryover was not present, matrix-matched samples containing no calibration standard material were injected, and blanks were injected following analysis of a known high-concentration sample (i.e., high level standards and QCs) and no carryover was detected.

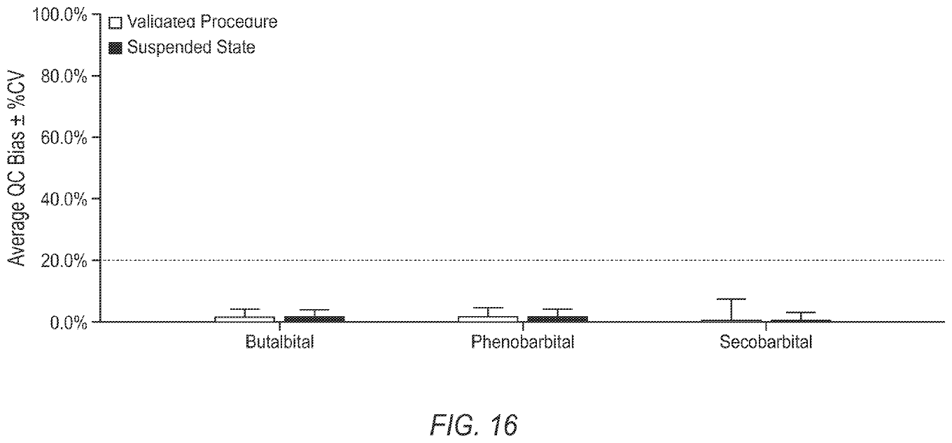

The LC-MS/MS method used for FIG. 16 incorporated 35 μl injections on a 2.6 μm Phenomenex Kinetex 2.6 (50×2.1 mm) LC column heated to 50° C. Analytes were resolved at 0.8 mL/min using mobile phase A (18.2 MΩ·cm water containing 10 mM acetic acid) and mobile phase B (100% methanol). Initial conditions were 35% mobile phase A/65% mobile phase B. A standard gradient that ramped to 25% mobile phase A/75% mobile phase B was used for all analyses in negative mode. The total run time including column equilibration period between injections was 4 min. Specific mass spectrometer and analyte parameters are specifically optimized for each analyte of interest. In general, two transitions were monitored for each analyte. Ion ratios were matched to those of calibration standards to ensure interfering metabolites and other compounds were resolved. To ensure carryover was not present, matrix-matched samples containing no calibration standard material were injected, and blanks were injected following analysis of a known high-concentration sample (i.e., high level standards and QCs) and no carryover was detected.

### Validation Study Design, Statistical Methods and Laboratory Accreditation Requirements

The PinPoint Testing, LLC laboratory is accredited to CLIA and ISO17025 standards. While the laboratory maintains an independent Quality Assurance/Quality Control program, method validation requirements follow criteria established by the Academy Standards Board for forensic laboratories (Standard 036 First Edition 2019), international standards typically used to regulate forensic and FDA laboratories (ISO17025), and CLIA standards established for clinical laboratories, disclosures of all of these validation methods and criteria are incorporated herein by reference in their entireties. When accuracy, precision, measurement of uncertainty, calibration model, reportable range, sensitivity, specificity, carryover, interference, ion suppression/enhancement, and analyte stability met required performance specifications the method validation was considered fit for forensic testing. Accuracy and precision were determined using QC samples prepared for independent experiments performed over non-consecutive days. Accuracy was calculated as bias for each of the expected QC concentrations. Within-run and between-run analytical precision was calculated as the coefficient of variance (% CV) for replicate measurements at three or four QC concentrations spanning the calibration range. The LOD was defined as the lowest calibrator level that could be confirmed through ion ratio comparisons. Coefficients of determination (r²) and residuals were calculated to assess the appropriateness of the calibration model. A minimum r²≥0.99 was required for passing validation.

1-45. (canceled)

46. A device for quantifying the concentration of at least one analyte in a liquid test sample, the device comprising:
a receptacle or plurality of receptacles, each receptacle configured to hold a liquid sample wherein each receptacle contains, respectively:
a) a matrix solution, and
b) a standard solution comprising an analyte standard selected from the group consisting of: a calibration standard, a quality control standard, a process control, an internal standard and a high temperature melting solvent; wherein the analyte standard further comprises one or more standards comprising an analyte that corresponds to the at least one analyte of the liquid test sample;
wherein the matrix solution is separated from the standard solution in said receptacle or plurality of receptacles, and the matrix solution and the standard solution are not in substantial admixture.

47. The device of claim 46, wherein the matrix solution and the standard solution are each independently separated frozen layers.

48. The device of claim 46, wherein the matrix solution and the standard solution are in suspended solid state.

49. The device of claim 46, wherein the matrix solution and the standard solution are separated by a barrier.

50. The device of claim 49, wherein the barrier comprises an air gap.

51. The device of claim 46, wherein the matrix solution comprises beta-glucuronidase enzyme and an enzyme buffer comprising an acetate buffer and a weak acid.

52. The device of claim 51, wherein the pH of the enzymatic reaction buffer ranges from about 3.5 to about 7.5.

53. The device of claim 46, wherein the matrix solution comprises 1,000 to 20,000 units of beta-glucoronidase.

54. The device of 53, wherein the matrix solution comprises betaglucoronidase isolated from: Patella vulgata, Helix aspersa, Helix pomatia, Abalone, purified recombinant systems, and mammalian liver.

55. The device of claim 46, wherein the standard solution comprises 0.1 ng/mL to 1 mg/mL of 6-MAM and a high temperature melting solvent.

56. The device of claim 55, wherein the high temperature melting solvent comprises DMSO, an alcohol, or an aqueous solution.

57. The device of claim 46, wherein the device comprises a multiwell plate having from 12 to 1536 wells.

58. The device of claim 57, wherein the multiwell plate has 96 wells.

59. The device of claim 57, wherein the device is a multiwell filtration plate configured to support at least one of liquid extraction (SLE), solid phase extraction (SPE), or liquid-liquid extraction (LLE).

60. The device of claim 46, wherein the receptacle or receptacles are constructed from a solid material selected from the group consisting of: metal, plastic or ceramic.

61. A system for the detection of an analyte in a human liquid sample, the system comprising:
a) a receptacle or plurality of receptacles, each receptacle configured to hold a human sample wherein each receptacle contains, respectively:
i) a matrix solution comprising blood, urine, oral fluid or beta-glucuronidase, and optionally an enzyme buffer solution, and
ii) a standard solution comprising an analyte standard selected from the group consisting of: a calibration standard, a quality control standard, a process control, an internal standard, and a high temperature melting solvent; wherein the matrix solution is separated from the standard solution in said receptacle or plurality of receptacles, and the matrix solution and the standard solution are not in substantial admixture in said receptacle or plurality of receptacles;
b) an analyte detection device operable to quantify the amount of said analyte in the human liquid sample relative to the amount of at least one analyte standard in a receptacle containing the human liquid sample;
wherein the amount of the analyte in the human liquid sample is detected with the analyte detection device after the standard solution and the matrix solution have mixed with the human liquid sample in the receptacle or plurality of receptacles.

62. The system of claim 61, wherein the matrix solution and the standard solution are each independently frozen layers.

63. The system of claim 61, wherein the matrix solution and the standard solution are at least one of separated by a barrier or in suspended solid state.

64. The system of claim 61, wherein the matrix solution comprises beta-glucuronidase enzyme and an enzyme buffer comprising an acetate buffer and a weak acid.

65. The system of claim 64, wherein the pH of the enzymatic buffer ranges from about 3.5 to about 7.5.

66. The system of claim 61, wherein the matrix solution comprises 1,000 to 20,000 units of beta-glucuronidase.

67. The system of claim 61, wherein the matrix solution comprises beta-glucuronidase isolated from: Patella vulgata, Helix aspersa, Helix pomatia, Abalone, purified recombinant enzyme and mammalian liver.

68. The system of claim 61, wherein the standard solution comprises 0.1 ng/mL to 1 mg/mL of cocaine, heroin, glucuronide conjugated drugs, methylphenedate, 6-MAM or a 6-MAM metabolite, and a high temperature melting solvent.

69. The system of claim 68, wherein the high temperature melting solvent comprises DMSO, an alcohol, or an aqueous solution.

70. The system of claim 61, wherein the device comprises a multiwell plate having from 12 to 1536 wells.

71. The system of claim 70, wherein the device is a multiwell plate having 96 wells.

72. The system of claim 61, wherein the receptacle is constructed from a solid material selected from the group consisting of: metal, plastic or ceramic.

73. The system of claim 61, wherein the device comprises a plurality of tubes, each tube capable of containing a volume of liquid ranging from 0.1 mL to about 100 mL.

74. The system of claim 61, wherein the analyte detection device comprises a mass spectrometer.

75. A method for measuring cocaine, heroin, glucuronide conjugated drugs, methylphenedate, 6-MAM or a 6-MAM metabolite; in a human biological sample, the method comprising:
(a) providing a device comprising:
(i) a plurality of receptacles, each receptacle configured to hold a liquid sample, wherein each receptacle contains, respectively:
(1) a matrix solution, and
(2) a standard solution comprising one of cocaine, heroin, glucuronide conjugated drugs, methylphenedate, 6-MAM or a 6-MAM metabolite; and
wherein the matrix solution is separated from the standard solution in said receptacle or plurality of receptacles, and the matrix solution and the standard solution are not in substantial admixture;
(b) mixing the matrix solution with the standard solution in each receptacle;
(c) adding a volume of the human biological sample to at least one receptacle in the device; and
(d) measuring the amount of cocaine, heroin, glucuronide conjugated drugs, methylphenedate, 6-MAM or a 6-MAM metabolite in the receptacles of the device.
